(19) United States
(12) Patent Application Publication
(10) Pub. No.: US 2010/0098746 A1
(43) Pub. Date: Apr. 22, 2010

(54) COMPOSITIONS AND METHODS FOR TREATING PERIODONTAL DISEASE COMPRISING CLONIDINE, SULINDAC AND/OR FLUOCINOLONE

(75) Inventor: Vanja Margareta King, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/572,387

(22) Filed: Oct. 2, 2009

Related U.S. Application Data
(60) Provisional application No. 61/106,815, filed on Oct. 20, 2008.

(51) Int. Cl. A61F 13/00; A61K 31/56

(52) U.S. Cl. 424/435; 514/171

(57) ABSTRACT

Effective treatments of periodontal disease for extended periods of time are provided. Through the administration of an effective amount of clonidine, sulindac, and/or fluocinolone at or near a target site, one can reduce, prevent, and/or treat periodontal disease. In some embodiments, when appropriate formulations are provided within biodegradable polymers, treatment can be continued for at least two weeks to two months.

### BACKGROUND

Periodontitis is a chronic inflammatory response caused by bacterial plaque that has spread below the gum line. Starting in the early stages as gingivitis, the later stages of periodontitis involves inflammation of the gums, connective tissues, and bones surrounding the teeth i.e. alveolar bones. Prolonged inflammation causes degenerative loss of tissues supporting the teeth and alveolar bone loss, eventually leading to loss of teeth. Tooth loss, caused by the loss of alveolar bone, is one of the major problems in clinical dentistry. In fact, periodontitis is the primary cause of tooth loss.

The periodontitis-associated inflammation occurring in the surrounding tissues that support the teeth is characterized by formation of infected “pockets” or spaces between the teeth and gums. These infected pockets contain debris, predominantly composed of microorganisms and their products (enzymes, endotoxins and other metabolic products), dental plaque, gingival fluid, food remnants, salivary mucin, desquamated epithelial cells, and leukocytes. Periodontal pockets are chronic inflammatory lesions, and as such are constantly undergoing repair. The condition of the soft tissue walls of the periodontal pocket results from a balance between destructive and constructive tissue changes. The destructive changes include the fluid and cellular inflammatory exudates and the associated degenerative changes stimulated by local bacterial infiltrate. The constructive changes include the formation of connective tissue cells, collagen fibers, and blood vessels in an effort to repair tissue damage caused by the inflammatory process. Healing does not go to completion because of the persistence of local irritants e.g., bacteria and the enzymes that they produce. These irritants stimulate fluid and cellular exudates, which in turn causes degeneration of the new tissue elements formed in the repair process. If purulent exudates are present in the infected pockets, it can contain living, degenerated and necrotic leukocytes (predominantly polymorphonuclear), living cells and dead bacterial cells, serum and a small amount of fibrin.

Conventional treatments for periodontal disease include scaling and root planing procedures. These procedures involve manually removing calculus, plaque and other deposits, smoothing the root surface to rid it of necrotic tooth substances, and curetting the inner surface of the gingival wall of the periodontal pockets to separate away any diseased soft tissue. The procedures aim to eliminate the infected pockets by reattaching connective tissue and epithelium to the tooth surface. By eliminating the environment for the microorganisms to grow, scaling and root planing procedures can successfully obliterate the infected pockets. These procedures may result in the replacement of diseased soft tissue with new soft tissue from growth and differentiation of new cells and intercellular substances. However, scaling and root planing procedures are ineffective in reducing the inflammatory process and stimulating the re-growth or replacement of destroyed bone and cementum caused by severe periodontitis. Often severe inflammation occurs in periodontitis leading to macrophage-like cells that enter the affected site that are eventually replaced with osteoclasts that breakdown bone leading to bone loss. Hence, there is a need for reliable or predictable compositions and methods to regenerate, augment, or restore alveolar bone loss and cementum loss inflicted by periodontitis.

Another initial treatment for periodontitis is administration of antibiotics, which may be optionally performed in conjunction with scaling and root planing procedures. Tetracycline, minocycline, amoxicillin or metronidazole may be used to remove the highly diverse populations of bacteria from the periodontal pockets. Despite the effectiveness of antibiotics in reducing inflammation and bacterial infection, antibiotic administration, like scaling and root planing, is also deficient in stimulating re-growth or replacement of the destroyed bone and cementum caused by severe periodontal disease.

When patients are not responsive to scaling and root planing procedures and/or antibiotic treatment, periodontal surgery, such as for example, gingivectomy or periodontal flap surgery is often required. In gingivectomy, the dentist reshapes the unhealthy gum tissue in order to reduce the size of the infected pocket. Reduction of the pocket size allows the patient to hygienically maintain the pocket by routine brushing and flossing, thereby eliminating a favorable environment for bacterial growth. Periodontal flap surgery is performed also when scaling and root planing procedures are unsuccessful, especially when there is loss of bone or tissue detachment. In this procedure, incisions are made in the gums and the surrounding alveolar bone is re-contoured to assist in healing of the infected area. Often times, surgical treatments are insufficient in stimulating re-growth or replacement of the destroyed bone and cementum caused by severe periodontal disease.

One pharmaceutical that is known to the medical profession is clonidine, which is widely recognized as an antihypertensive agent that acts as an agonist on the alpha-2-adrenergic receptor and as a neural receptor agonist. In general, clonidine, also referred to as 2,6-dichloro-N-2-imidazolidinyldenebenzenamine (C₉H₉Cl₂N₃) may be represented by the following chemical structure:

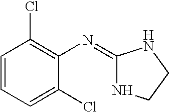

Another pharmaceutical that is known to the medical profession is sulindac, (commercially available as Clinoril® from Sigma as a free acid) which is widely recognized as a non-steroidal anti-inflammatory of the arylalkanoic acid class. It may be represented by the following chemical formula C20H17FO3S. Sulindac is a prodrug, derived from sulfinylindene that is converted in the body to an active NSAID (non-steroidal anti-inflammatory agent). More specifically, the agent is converted by liver enzymes to a sulfide that is excreted in the bile and then reabsorbed from the intestine. This is thought to help maintain constant blood levels with reduced gastrointestinal side effects.

Yet another pharmaceutical that is known to reduce inflammation is fluocinolone, which in its acetonide form (C₂₄H₃₀F₂O₆) has been administered topically as a cream to treat skin inflammation. It may also be referred to as 4b,12-Difluoro-6b-glycoloyl-5-hydroxy-4a,6a,8,8-tetramethyl-4a,4b,5,6,6a,6b,9a,10,10a,10b,11,12-dodecahydro-2H-naphtho[2′, 1′:4,5]indeno[1,2-d][1,3]dioxol-2-one or 6α-,9α-Difluoro-16 α-hydroxyprednisolone 16,17-acetonide.

However, to date clonidine, sulindac, and/or fluocinolone have not been appreciated as an effective treatment for periodontal disease in sustained release formulations that provide treatment over at least 2 weeks. Thus, there is a need to develop effective formulations of clonidine, sulindac, and/or fluocinolone for reducing, preventing or treating periodontal disease.

### SUMMARY

Compositions and methods are provided comprising clonidine, sulindac, and/or fluocinolone or its pharmaceutically acceptable salts or esters thereof that are administered in order to reduce, prevent or treat periodontal disease.

In some embodiments, administering anti-inflammatory agents such as clonidine, sulindac, and/or fluocinolone, inflammatory cytokines are down regulated and osteoclast differentiation and bone resorption decreased, enhancing the quality of bone formation in the oral cavity, which is beneficial in the treatment of periodontal disease.

In some embodiments, an implantable drug depot is provided for reducing, preventing or treating periodontal disease in a patient in need of such treatment, the implantable drug depot comprising clonidine in an amount from about 1 wt. % to about 20 wt. % of the drug depot, fluocinolone in an amount from about 0.05 wt. % to about 25 wt. % of the drug depot, and/or sulindac in an amount from about 20 wt. % to about 40 wt. % of the drug depot and at least one biodegradable polymer, wherein the drug depot is capable of releasing clonidine, fluocinolone and/or sulindac over a period of at least two weeks.

In some embodiments, an implantable drug depot is provided for reducing, preventing or treating periodontal disease in a patient in need of such treatment, the implantable drug depot comprising clonidine hydrochloride in an amount of from about 1 wt. % to about 20 wt. % of the drug depot, fluocinolone acetonide in an amount from about 0.05 wt. % to about 25 wt. % of the drug depot, and/or sulindac sodium in an amount from about 20 wt. % to about 40 wt. % of the drug depot and at least one polymer, wherein the at least one polymer comprises one or more of poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone or a combination thereof.

In some embodiments, a method is provided for treating periodontal disease in a patient in need of such treatment, the method comprises implanting into an oral cavity of the patient a drug depot comprising clonidine in an amount of from about 1 wt. % to about 20 wt. % of the drug depot, fluocinolone in an amount from about 0.05 wt. % to about 25 wt. % of the drug depot, and/or sulindac in an amount from about 20 wt. % to about 40 wt. % of the drug depot and at least one biodegradable polymer, wherein the drug depot is capable of releasing clonidine, fluocinolone and/or sulindac over a period of at least two weeks, thereby treating the periodontal disease.

According to one embodiment, there is a pharmaceutical formulation comprising: clonidine in an amount from about 1 wt. % to about 20 wt. % of the formulation, fluocinolone in an amount from about 0.05 wt. % to about 25 wt. % of the formulation, and/or sulindac in an amount from about 20 wt. % to about 40 wt. % of the formulation, and at least one biodegradable polymer. The pharmaceutical composition may for example, be part of a drug depot. The drug depot may: (i) consist of only the drug(s) (or one or more of its pharmaceutically acceptable salts) and the biodegradable polymer(s); or (ii) consist essentially of the drug(s) (or one or more of its pharmaceutically acceptable salts) and the biodegradable polymer(s); or (iii) comprise the drug(s) (or one or more of its pharmaceutically acceptable salts), the biodegradable polymer(s) and one or more other active ingredients, surfactants, excipients or other ingredients or combinations thereof. When there are other active ingredients, surfactants, excipients or other ingredients or combinations thereof in the formulation, in some embodiments these other compounds or combinations thereof comprise less than 20 wt. %, less than 19 wt. %, less than 18 wt. %, less than 17 wt. %, less than 16 wt. %, less than 15 wt. %, less than 14 wt. %, less than 13 wt. %, less than 12 wt. %, less than 11 wt. %, less than 10 wt. %, less than 9 wt. %, less than 8 wt. %, less than 7 wt. %, less than 6 wt. %, less than 5 wt. %, less than 4 wt. %, less than 3 wt. %, less than 2 wt. %, less than 1 wt. % or less than 0.5 wt. %.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 illustrates the effect of clonidine on osteoclast differentiation and resorption. Clonidine significantly decreased osteoclast differentiation and bone resorption at 100 μM and 10 μM when compared to the RANK (receptor activator or NF_{κ}β) only treated control wells. In this study, clonidine decreased osteoclast differentiation and thus inflammation and also slowed down bone resorption, both of which is helpful in periodontal disease.

FIG. 2 illustrates the effect of sulindac on osteoclast differentiation and resorption. Sulindac significantly decreased osteoclast differentiation and resorption at 140.3 μM when compared to the RANK only treated control wells. In this study, sulindac decreased osteoclast differentiation and thus inflammation and also slowed down bone resorption, both of which is helpful in periodontal disease.

FIG. 3 is a graphic representation of inflammation assessment following treatment of the minipigs with injected drug on days 1, 3 and 4 post-surgery. High dose clonidine (150 μg) had a very low inflammation score. The low dose clonidine given was 75 μg.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

### DEFINITIONS

It is noted that, as used in this specification and the appended claims, the singular forms “a,” “an,” and “the,” include plural referents unless expressly and unequivocally limited to one referent. Thus, for example, reference to “a drug depot” includes one, two, three or more drug depots.

“Periodontal disease” includes any condition that affects the gums and other structures supporting the teeth. The most common form of periodontal disease is caused by bacterial infections. These bacteria grow in a sticky film called dental plaque that sticks on the tooth surfaces next to the gums. The bacteria can cause inflammation, spread and destroy the gums and the supporting bone around the teeth. The mildest form of periodontal disease is gingivitis, which affects only the gums. More severe periodontal disease damages the other supporting structures of the bone and/or tooth.

The terms “oral tissue” or “oral cavity” includes tissue within the orofacial environment and includes tissue sites located within the orofacial environment. Such tissue includes by way of illustration and not limitation, periodontal tissue such as the periodontium; periodontal ligaments; bone tissue, bone tissue at the end of an infected tooth, inside the tooth or within the bone cavity such as may be present after an apicoectomy or tooth extraction; endodontic tissue; bone tissue surrounding an implant fixture; jaw tissue such as the temporomandibular joint, the temporalis muscle, the temporal bone the masseter muscle and the mandible; tissue affected by surgery, e.g. alveolar ridge augmentation, or so forth.

The terms “intratissue” and “intraorofacial tissue” are used interchangeably and are intended to mean that the controlled release drug delivery system is implanted inside the orofacial tissue in contrast to topically. The drug delivery system can be implanted within the tissue by a punch biopsy procedure, inserted into the tissue with a trocar, inserted into the tissue after a surgical incision, left in the open wound after a surgical procedure, and so forth. The term intratissue is intended to include intraosseous. In some embodiments, the drug depot can be administered via supratis sue placement such as on top of the gum tissue or intersulcular placement such as on top of the periodontal pocket.

The term “target tissue site” or “implant site” is intended to mean the intratissue location of the drug delivery system. The “target site” is the location of the tissue to be treated. Typically the implant site will be the same as the target site to provide for optimal targeted drug delivery. However, the present application also contemplates positioning the drug depot at a placement site nearby the target site such that the therapeutic agent can be delivered to the surrounding vasculature, which carries the agent to the desired nearby target site.

A “drug depot” is the composition in which the clonidine, sulindac and/or fluocinolone is administered to the body. Thus, a drug depot may comprise a physical structure to facilitate implantation and retention in a desired site (e.g., gum, bone, muscle, etc.). The drug depot may also comprise the drug itself. The term “drug” as used herein is generally meant to refer to any substance that alters the physiology of a patient. The term “drug” may be used interchangeably herein with the terms “therapeutic agent,” “therapeutically effective amount,” and “active pharmaceutical ingredient” or “API.” It will be understood that unless otherwise specified a “drug” formulation may include more than one therapeutic agent, wherein exemplary combinations of therapeutic agents include a combination of two or more drugs. The drug provides a concentration gradient of the therapeutic agent for delivery to the site. In various embodiments, the drug depot provides an optimal drug concentration gradient of the therapeutic agent at a distance of up to about 1 cm to about 5 cm from the administration site and comprises clonidine, sulindac, and/or fluocinolone. A drug depot may also include a pump or pellet.

A “therapeutically effective amount” or “effective amount” is such that when administered, the drug results in alteration of the biological activity, such as, for example, inhibition of inflammation, reduction or alleviation of periodontal disease, etc. The dosage administered to a patient can be as single or multiple doses depending upon a variety of factors, including the drug's administered pharmacokinetic properties, the patient's conditions and characteristics (sex, age, body weight, health, size, etc.), extent of symptoms, concurrent treatments, frequency of treatment and the effect desired. In some embodiments the formulation is designed for immediate release. In other embodiments the formulation is designed for sustained release. In other embodiments, the formulation comprises one or more immediate release surfaces and one or more sustained release surfaces.

A “depot” includes but is not limited to capsules, microspheres, microparticles, microcapsules, microfibers particles, nanospheres, nanoparticles, coating, matrices, wafers, pills, pellets, emulsions, liposomes, micelles, gels, or other pharmaceutical delivery compositions or a combination thereof. Suitable materials for the depot are ideally pharmaceutically acceptable biodegradable and/or any bioabsorbable materials that are preferably FDA approved or GRAS materials. These materials can be polymeric or non-polymeric, as well as synthetic or naturally occurring, or a combination thereof.

The term “biodegradable” includes that all or parts of the drug depot will degrade over time by the action of enzymes, by hydrolytic action and/or by other similar mechanisms in the human body. In various embodiments, “biodegradable” includes that the depot (e.g., microparticle, microsphere, etc.) can break down or degrade within the body to non-toxic components after or while a therapeutic agent has been or is being released. By “bioerodible” it is meant that the depot will erode or degrade over time due, at least in part, to contact with substances found in the surrounding tissue, fluids or by cellular action. By “bioabsorbable” it is meant that the depot will be broken down and absorbed within the human body, for example, by a cell or tissue. “Biocompatible” means that the depot will not cause substantial tissue irritation or necrosis at the target tissue site.

The phrases “sustained release” and “sustain release” (also referred to as extended release or controlled release) are used herein to refer to one or more therapeutic agent(s) that is introduced into the body of a human or other mammal and continuously or continually releases a stream of one or more therapeutic agents over a predetermined time period and at a therapeutic level sufficient to achieve a desired therapeutic effect throughout the predetermined time period. Reference to a continuous or continual release stream is intended to encompass release that occurs as the result of biodegradation in vivo of the drug depot, or a matrix or component thereof, or as the result of metabolic transformation or dissolution of the therapeutic agent(s) or conjugates of therapeutic agent(s).

The phrase “immediate release” is used herein to refer to one or more therapeutic agent(s) that is introduced into the body and that is allowed to dissolve in or become absorbed at the location to which it is administered, with no intention of delaying or prolonging the dissolution or absorption of the drug.

The two types of formulations (sustain release and immediate release) may be used in conjunction. The sustained release and immediate release may be in one or more of the same depots. In various embodiments, the sustained release and immediate release may be part of separate depots. For example, a bolus or immediate release formulation of clonidine, sulindac, and/or fluocinolone may be placed at or near the target site and a sustain release formulation may also be placed at or near the same site. Thus, even after the bolus becomes completely accessible, the sustain release formulation would continue to provide the active ingredient for the intended tissue.

In various embodiments, the drug depot can be designed to cause an initial burst dose of therapeutic agent within the first twenty-four hours to forty-eight hours after implantation.

“Initial burst” or “burst effect” or “bolus dose” refers to the release of therapeutic agent from the depot during the first twenty-four hours after the depot comes in contact with an aqueous fluid (e.g., blood circulating in the oral cavity, saliva, etc.). The “burst effect” is believed to be due to the increased release of therapeutic agent from the depot. In alternative embodiments, the depot (e.g., gel) is designed to avoid this initial burst effect.

“Treating” or “treatment” of a disease or condition refers to executing a protocol that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes protocols that have only a marginal effect on the patient. Treatment can include inhibiting the disease, e.g., arresting its development; or relieving the disease, e.g., causing regression of the disease. For example, treatment can include reducing acute or chronic inflammation; alleviating pain and mitigating and inducing re-growth of new periodontal ligament, bone and other tissues; as an adjunct in orthognathic surgery; any elective cosmetic surgical or repair procedure; and so forth.

“Localized” delivery includes delivery where one or more drugs are deposited within a tissue, for example, an alveolar ridge bone tissue in the oral cavity, or in close proximity (within about 0.1 cm, or preferably within about 10 cm, for example) thereto.

The term “mammal” refers to organisms from the taxonomy class “mammalian,” including but not limited to humans, other primates such as chimpanzees, apes, orangutans and monkeys, rats, mice, cats, dogs, cows, horses, etc.

The phrase “release rate profile” refers to the percentage of active ingredient that is released over fixed units of time, e.g., mcg/hr, mcg/day, 10% per day for ten days, etc. As persons of ordinary skill know, a release rate profile may, but need not, be linear. By way of a non-limiting example, the drug depot may be a ribbon-like fiber that releases the clonidine over a period of time.

The term “solid” is intended to mean a rigid material, while, “semi-solid” is intended to mean a material that has some degree of flexibility, thereby allowing the depot to bend and conform to the surrounding tissue requirements.

The abbreviation “DLG” refers to poly(DL-lactide-co-glycolide).

The abbreviation “DL” refers to poly(DL-lactide).

The abbreviation “LG” refers to poly(L-lactide-co-glycolide).

The abbreviation “CL” refers to polycaprolactone.

The abbreviation “DLCL” refers to poly(DL-lactide-co-caprolactone).

The abbreviation “LCL” refers to poly(L-lactide-co-caprolactone).

The abbreviation “G” refers to polyglycolide.

The abbreviation “PEG” refers to poly(ethylene glycol).

The abbreviation “PLGA” refers to poly(lactide-co-glycolide) also known as poly(lactic-co-glycolic acid), which are used interchangeably.

The abbreviation “PLA” refers to polylactide.

The abbreviation “POE” refers to poly(orthoester).

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

The headings below are not meant to limit the disclosure in any way; embodiments under any one heading may be used in conjunction with embodiments under any other heading.

Inflammatory actions of inflammatory cytokines such as tumor necrosis factor (TNF), interleukins (IL), and other inflammatory cytokines, has been shown to increase osteoclast formation and decrease bone resorption leading to poor bone quality. In periodontal disease, prolonged inflammation causes degenerative loss of tissues supporting the teeth and alveolar bone loss, eventually leading to loss of teeth. Thus, there is a great need to treat periodontal disease in a way that both slows down the body's response (inflammation) and treats the results of the inflammation (bone resorption).

The inventor finds that by administering anti-inflammatory agents such as clonidine, sulindac, and/or fluocinolone, inflammatory cytokines, such as TNF, IL and other cytokines are down regulated and osteoclast differentiation and bone resorption decreased, enhancing the quality of bone formation. Further, the result of severe periodontal disease is a loss of the bone that holds the teeth in place. This is an “unintended” consequence of the inflammation, and causes significant problems. The anti-inflammatory agent, decreases, blocks, inhibits, abrogates or interferes with the inflammatory cascade leading to improved bone growth and quality bone is produced beneficial in reducing, preventing and/or treating periodontal disease.

In some embodiments, the decrease in osteoclast differentiation and bone resorption is at least 5%, 10%, 20%, 30%, 40%, 50%, 60%, 70%, 80%, 90%, 95% or 99% decreased when compared to in vitro or in vivo systems that are not treated with anti-inflammatory agents (e.g., clonidine, sulindac, fluocinolone, etc.)

The clonidine, sulindac, and/or fluocinolone can be administered alone, or before, during or after conventional periodontal treatments, such as for example, scaling and root planing procedures and/or antibiotic treatment, periodontal surgery, such as for example, gingivectomy or periodontal flap surgery or the like.

### Clonidine

When referring to clonidine, unless otherwise specified or apparent from context it is understood that the inventor is also referring to pharmaceutically acceptable salts and/or esters thereof. One well-known commercially available salt for clonidine is its hydrochloride salt. Some other examples of potentially pharmaceutically acceptable salts include those salt-forming acids and bases that do not substantially increase the toxicity of a compound, such as, salts of alkali metals such as magnesium, potassium and ammonium, salts of mineral acids such as hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids, and the like.

Further, when referring to clonidine the active ingredient may not only be in the salt form, but also in the base form (e.g., free base). In various embodiments, if it is in the base form, it may be combined with polymers under conditions in which there is not severe polymer degradation, as may be seen upon heat or solvent processing that may occur with PLGA or PLA. By way of a non limiting example, when formulating clonidine with poly(orthoesters) it may be desirable to use the clonidine base formulation. By contrast, when formulating clonidine with PLGA, it may be desirable to use the HCl salt form.

Suitable clonidine drug depot formulations for use in the present application are described in U.S. Patent Application Ser. No. 61/046,201 filed Apr. 18, 2008, and U.S. patent application Ser. No. 12/105,474, filed Apr. 18, 2008, the entire disclosures of which are herein incorporated by reference.

### Sulindac

When referring to sulindac, unless otherwise specified or apparent from context it is understood that the inventor is also referring to pharmaceutically acceptable salts, pharmacologically-active derivatives of the sulindac or an active metabolite of the sulindac. As used herein, “pharmaceutically acceptable salts” refer to derivatives of the disclosed compounds (e.g., esters or amines) wherein the parent compound may be modified by making acidic or basic salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, or nitric acids; or the salts prepared from organic acids such as acetic, fuoric, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acid. Pharmaceutically acceptable also includes the racemic mixtures ((+)—R and (−)—S enantiomers) or each of the dextro and levo isomers of the sulindac individually. The sulindac may be in the free acid or base form or be pegylated for long acting activity.

One well-known commercially available salt for sulindac is its sodium salt (e.g., available from Spectrum Chemical) or sulfide salt.

Suitable sulindac drug depot formulations for use in the present application are described in U.S. Patent Application Ser. No. 61/046,246, filed Apr. 18, 2008, the entire disclosure of which is herein incorporated by reference.

### Fluocinolone

When referring to fluocinolone, unless otherwise specified or apparent from context it is understood that the inventor is also referring to pharmaceutically acceptable salts, pharmacologically-active derivatives of the fluocinolone or an active metabolite of the fluocinolone. As used herein, “pharmaceutically acceptable salts” refer to derivatives of the disclosed compounds (e.g., esters or amines) wherein the parent compound may be modified by making acidic or basic salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, or nitric acids; or the salts prepared from organic acids such as acetic, fuoric, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acid. Pharmaceutically acceptable also includes the racemic mixtures ((+)—R and (−)—S enantiomers) or each of the dextro and levo isomers of the fluocinolone individually. The fluocinolone may be in the free acid or base form or be pegylated for long acting activity.

One common form of fluocinolone for administration to mammals is fluocinolone acetonide. Suitable fluocinolone drug depot formulations for use in the present application are described in U.S. Patent Application Ser. No. 61/046,218, filed Apr. 18, 2008, the entire disclosure of which is herein incorporated by reference.

The clonidine, sulindac, and/or fluocinolone or its pharmaceutically acceptable salt or esters thereof may be administered with a muscle relaxant. Exemplary muscle relaxants include by way of example and not limitation, alcuronium chloride, atracurium bescylate, baclofen, carbamate, carbolonium, carisoprodol, chlorphenesin, chlorzoxazone, cyclobenzaprine, dantrolene, decamethonium bromide, fazadinium, gallamine triethiodide, hexafluorenium, meladrazine, mephensin, metaxalone, methocarbamol, metocurine iodide, pancuronium, pridinol mesylate, styramate, suxamethonium, suxethonium, thiocolchicoside, tizanidine, tolperisone, tubocuarine, vecuronium, or combinations thereof.

The drug depot may comprise other therapeutic agents in addition to the clonidine, sulindac and/or fluocinolone as well. These therapeutic agents, in various embodiments, block the transcription or translation of TNF-α or other proteins in the inflammation cascade. Suitable therapeutic agents include, but are not limited to, integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibodies (daclizumab, basilicimab), ABX (anti IL-8 antibodies), recombinant human IL-10, or HuMax IL-15 (anti-IL 15 antibodies).

Other suitable therapeutic agents include IL-1 inhibitors, such Kineret® (anakinra) which is a recombinant, non-glycosylated form of the human inerleukin-1 receptor antagonist (IL-1Ra), or AMG 108, which is a monoclonal antibody that blocks the action of IL-1. Therapeutic agents also include excitatory amino acids such as glutamate and aspartate, antagonists or inhibitors of glutamate binding to NMDA receptors, AMPA receptors, and/or kainate receptors. Interleukin-1 receptor antagonists, thalidomide (a TNF-αrelease inhibitor), thalidomide analogues (which reduce TNF-α production by macrophages), bone morphogenetic protein (BMP) type 2 and BMP-4 (inhibitors of caspase 8, a TNF-α activator), quinapril (an inhibitor of angiotensin II, which upregulates TNF-α), interferons such as IL-11 (which modulate TNF-α receptor expression), and aurin-tricarboxylic acid (which inhibits TNF-α), may also be useful as therapeutic agents for reducing inflammation. It is further contemplated that where desirable a pegylated form of the above may be used. Examples of still other therapeutic agents include NF kappa B inhibitors such as glucocorticoids, antioxidants, such as dithiocarbamate, and other compounds, such as, for example, sulfasalazine.

Examples of therapeutic agents suitable for use also include, but are not limited to an anti-inflammatory agent, an analgesic agent, or an osteoinductive growth factor or an anti-infective agent (e.g., antiviral, antibacterial, antifungal agents, etc.), or a combination thereof. Anti-inflammatory agents include, but are not limited to, apazone, celecoxib, diclofenac, diflunisal, enolic acids (piroxicam, meloxicam), etodolac, fenamates (mefenamic acid, meclofenamic acid), gold, ibuprofen, indomethacin, ketoprofen, ketorolac, nabumetone, naproxen, nimesulide, salicylates, sulfasalazine[2-hydroxy-5-[4-[C2-pyridinylamino)sulfonyl]azo]benzoic acid, tepoxalin or tolmetin; as well as antioxidants, such as dithiocarbamate, steroids, such as cortisol, cortisone, hydrocortisone, fludrocortisone, prednisone, prednisolone, methylprednisolone, triamcinolone, betamethasone, dexamethasone, beclomethasone, fluticasone or a combination thereof.

Suitable anabolic growth or anti-catabolic growth factors include, but are not limited to, a bone morphogenetic protein, a growth differentiation factor, a LIM mineralization protein, CDMP or progenitor cells or a combination thereof.

Suitable analgesic agents include, but are not limited to, acetaminophen, bupivacaine, lidocaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, mepiridine, methadone, morphine, nalbuphine, opium, oxycodone, papavereturn, pentazocine, pethidine, phenoperidine, piritramide, dextropropoxyphene, remifentanil, tilidine, tramadol, codeine, dihydrocodeine, meptazinol, dezocine, eptazocine, flupirtine, amitriptyline, carbamazepine, gabapentin, pregabalin, or a combination thereof.

Exemplary anti-infective agents by way of example and not limitation, antibacterial agents; quinolones and in particular fluoroquinolones (e.g., norfloxacin, ciprofloxacin, lomefloxacin, ofloxacin, etc.), aminoglycosides (e.g., gentamicin, tobramycin, etc.), glycopeptides (e.g., vancomycin, etc.), lincosamides (e.g., clindamycin), cephalosporins (e.g., first, second, third generation) and related beta-lactams, macrolides (e.g., azithromycin, erythromycin, etc.), nitroimidazoles (e.g., metronidazole), penicillins, polymyxins, tetracyclines, or combinations thereof.

Other exemplary antibacterial agents include, by way of illustration and not limitation, acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; zorbamycin; or combinations thereof.

The clonidine, sulindac and/or fluocinolone may also be administered with non-active ingredients. These non-active ingredients may have multi-functional purposes including the carrying, stabilizing and controlling the release of the therapeutic agent(s). The sustained release process, for example, may be by a solution-diffusion mechanism or it may be governed by an erosion-sustained process. Typically, the depot will be a solid or semi-solid formulation comprised of a biocompatible material that can be biodegradable.

Exemplary excipients that may be formulated with clonidine, sulindac, and/or fluocinolone in addition to the biodegradable polymer include but are not limited to MgO (e.g., 1 wt. %), 5050 DLG 6E, 5050 DLG 1A, mPEG, TBO—Ac, mPEG, Span-65, Span-85, pluronic F127, TBO—Ac, sorbital, D-sorbitol, cyclodextrin, B-cyclodextrin, maltodextrin, pluronic F68, CaCl, 5050 7A MgCO₃, paraffin oil, barium sulfate, paraffin oil, glycerol monooleate, tributyl-ortho-acetylcitrate (CAS: 77-90-7) (TBO-ac) or PEG 1500, Pluronic F68, 5050 PLG 7A or combinations thereof. In some embodiments, the excipients comprise from about 0.001 wt. % to about 50 wt. % of the formulation. In some embodiments, the excipients comprise from about 0.001 wt. % to about 40 wt. % of the formulation. In some embodiments, the excipients comprise from about 0.001 wt. % to about 30 wt. % of the formulation. In some embodiments, the excipients comprise from about 0.001 wt. % to about 20 wt. % of the formulation. In some embodiments, the excipients comprise from about 0.001 wt. % to about 10 wt. % of the formulation. In some embodiments, the excipients comprise from about 0.001 wt. % to about 50 wt. % of the formulation. In some embodiments, the excipients comprise from about 0.001 wt. % to about 2 wt. % of the formulation.

In various embodiments, the non-active ingredients will be durable within the tissue site for a period of time equal to (for biodegradable components) or greater than (for non-biodegradable components) the planned period of drug delivery. For example, the depot material may have a melting point or glass transition temperature close to or higher than body temperature, but lower than the decomposition or degradation temperature of the therapeutic agent. However, the pre-determined erosion of the depot material can also be used to provide for slow release of the loaded therapeutic agent(s). Non-biodegradable polymers include but are not limited to PVC and polyurethane.

In some embodiments, the drug depot may not be biodegradable. For example, the drug depot may comprise polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof. Typically, these types of drug depots may need to be removed after a certain amount of time.

In some instances, it may be desirable to avoid having to remove the drug depot after use. In those instances, the depot may comprise a biodegradable material. There are numerous materials available for this purpose and having the characteristic of being able to breakdown or disintegrate over a prolonged period of time when positioned at or near the target tissue. As a function of the chemistry of the biodegradable material, the mechanism of the degradation process can be hydrolytical or enzymatical in nature, or both. In various embodiments, the degradation can occur either at the surface (heterogeneous or surface erosion) or uniformly throughout the drug delivery system depot (homogeneous or bulk erosion).

In various embodiments, the depot may comprise a bioerodable, a bioabsorbable, and/or a biodegradable biopolymer that may provide immediate release, or sustained release of the clonidine. Examples of suitable sustained release biopolymers include but are not limited to poly(alpha-hydroxy acids), poly(lactide-co-glycolide) (PLGA), polylactide (PLA), polyglycolide (PG), polyethylene glycol (PEG) conjugates of poly(alpha-hydroxy acids), poly(orthoester)s (POE), polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) or combinations thereof. As persons of ordinary skill are aware, mPEG may be used as a plasticizer for PLGA, but other polymers/excipients may be used to achieve the same effect. mPEG imparts malleability to the resulting formulations.

In various embodiments, the drug depot comprises poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone or a combination thereof.

As persons of ordinary skill in the art are aware, an implantable depot compositions having a blend of polymers with different end groups are used the resulting formulation will have a lower burst index and a regulated duration of delivery. For example, one may use polymers with acid (e.g., carboxylic acid) and ester end groups (e.g., methyl of ethyl ester end groups).

Additionally, by varying the comonomer ratio of the various monomers that form a polymer (e.g., the L/G (lactic acid/glycolic acid) or G/CL (glycolic acid/polycaprolactone) ratio for a given polymer) there will be a resulting depot composition having a regulated burst index and duration of delivery. For example, a depot composition having a polymer with a L/G ratio of 50:50 may have a short duration of delivery ranging from about two days to about one month; a depot composition having a polymer with a L/G ratio of 65:35 may have a duration of delivery of about two months; a depot composition having a polymer with a L/G ratio of 75:25 or L/CL ratio of 75:25 may have a duration of delivery of about three months to about four months; a depot composition having a polymer ratio with a L/G ratio of 85:15 may have a duration of delivery of about five months; a depot composition having a polymer with a L/CL ratio of 25:75 or PLA may have a duration of delivery greater than or equal to six months; a depot composition having a terpolymer of CL/G/L with G greater than 50% and L greater than 10% may have a duration of delivery of about one month and a depot composition having a terpolymer of CL/G/L with G less than 50% and L less than 10% may have a duration months up to six months. In general, increasing the G content relative to the CL content shortens the duration of delivery whereas increasing the CL content relative to the G content lengthens the duration of delivery. Thus, among other things, depot compositions having a blend of polymers having different molecular weights, end groups and comonomer ratios can be used to create a depot formulation having a lower initial burst and a regulated duration of delivery.

The depot may optionally contain inactive materials such as buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate, calcium or calcium salts such as calcium carbonate, calcium phosphate; degradation/release modifiers; drug release adjusting agents; emulsifiers; preservatives such as benzalkonium chloride, chlorobutanol, phenylmercuric acetate and phenylmercuric nitrate, sodium bisulfate, sodium bisulfite, sodium thiosulfate, thimerosal, methylparaben, polyvinyl alcohol and phenylethyl alcohol; solubility adjusting agents; stabilizers; and/or cohesion modifiers.

The depot can be different sizes, shapes and configurations. There are several factors that can be taken into consideration in determining the size, shape and configuration of the drug depot. For example, both the size and shape may allow for ease in positioning the drug depot at the target tissue site that is selected as the implantation or injection site. In addition, the shape and size of the system should be selected so as to minimize or prevent the drug depot from moving after implantation or injection. In various embodiments, the drug depot can be shaped like a sphere, a cylinder such as a rod or fiber, a flat surface such as a disc, film or sheet (e.g., ribbon-like) or the like. Flexibility may be a consideration so as to facilitate placement of the drug depot. In various embodiments, the drug depot can be different sizes, for example, the drug depot may be a length of from about 0.5 mm to 5 mm and have a diameter of from about 0.01 to about 2 mm. In various embodiments, the drug depot may have a layer thickness of from about 0.005 to 1.0 mm, such as, for example, from 0.05 to 0.75 mm.

Radiographic markers can be included on the drug depot to permit the user to position the depot accurately into the target site of the patient. These radiographic markers will also permit the user to track movement and degradation of the depot at the site over time. In this embodiment, the user may accurately position the depot in the site using any of the numerous diagnostic imaging procedures. Such diagnostic imaging procedures include, for example, X-ray imaging. Examples of such radiographic markers include, but are not limited to, barium, calcium phosphate, and/or metal beads or particles. In various embodiments, the radiographic marker could be a spherical shape, a ring around the depot, or a coating.

### Gel

In various embodiments, the clonidine, sulindac, and/or fluocinolone is administered in a gel. The gel may have a pre-dosed viscosity in the range of about 1 to about 500 centipoise (cps), 1 to about 200 cps, or 1 to about 100 cps. After the gel is administered to the target site, the viscosity of the gel will increase and the gel will have a modulus of elasticity (Young's modulus) in the range of about 1×10⁴ to about 6×10⁵ dynes/cm², or 2×10⁴ to about 5×10⁵ dynes/cm², or 5×10⁴ to about 5×10⁵ dynes/cm².

In one embodiment, a depot comprises an adherent gel comprising clonidine, sulindac, and/or fluocinolone that can be evenly distributed throughout the gel. The gel may be of any suitable type, as previously indicated, and should be sufficiently viscous so as to prevent the gel from migrating from the targeted delivery site once deployed; the gel should, in effect, “stick” or adhere to the targeted tissue site. The gel may, for example, solidify upon contact with the targeted tissue or after deployment from a targeted delivery system. The targeted delivery system may be, for example, a syringe, a catheter, needle or cannula or any other suitable device. The targeted delivery system may inject the gel into or on the targeted tissue site. The therapeutic agent may be mixed into the gel prior to the gel being deployed at the targeted tissue site. In various embodiments, the gel may be part of a two-component delivery system and when the two components are mixed, a chemical process is activated to form the gel and cause it to stick or to adhere to the target tissue.

In various embodiments, a gel is provided that hardens or stiffens after delivery. Typically, hardening gel formulations may have a pre-dosed modulus of elasticity in the range of about 1×10⁴ to about 3×10⁵ dynes/cm², or 2×10⁴ to about 2×10⁵ dynes/cm², or 5×10⁴ to about 1×10⁵ dynes/cm². The post-dosed hardening gels (after delivery) may have a rubbery consistency and have a modulus of elasticity in the range of about 1×10⁴ to about 2×10⁶ dynes/cm², or 1×10⁵ to about 7×10⁵ dynes/cm², or 2×10⁵ to about 5×10⁵ dynes/cm².

In various embodiments, for those gel formulations that contain a polymer, the polymer concentration may affect the rate at which the gel hardens (e.g., a gel with a higher concentration of polymer may coagulate more quickly than gels having a lower concentration of polymer). In various embodiments, when the gel hardens, the resulting matrix is solid but is also able to conform to the irregular surface of the tissue (e.g., recesses and/or projections in bone).

The percentage of polymer present in the gel may also affect the viscosity of the polymeric composition. For example, a composition having a higher percentage by weight of polymer is typically thicker and more viscous than a composition having a lower percentage by weight of polymer. A more viscous composition tends to flow more slowly. Therefore, a composition having a lower viscosity may be preferred in some instances. In some embodiments, the polymer comprises 20 wt. % to 90 wt. % of the formulation.

In various embodiments, the molecular weight of the gel can be varied by many methods known in the art. The choice of method to vary molecular weight is typically determined by the composition of the gel (e.g., polymer, versus non-polymer). For example in various embodiments, when the gel comprises one or more polymers, the degree of polymerization can be controlled by varying the amount of polymer initiators (e.g. benzoyl peroxide), organic solvents or activator (e.g. DMPT), crosslinking agents, polymerization agent, and/or reaction time.

Suitable gel polymers may be soluble in an organic solvent. The solubility of a polymer in a solvent varies depending on the crystallinity, hydrophobicity, hydrogen-bonding and molecular weight of the polymer. Lower molecular weight polymers will normally dissolve more readily in an organic solvent than high-molecular weight polymers. A polymeric gel that includes a high molecular weight polymer tends to coagulate or solidify more quickly than a polymeric composition that includes a low-molecular weight polymer. Polymeric gel formulations that include high molecular weight polymers, also tend to have a higher solution viscosity than a polymeric gel that includes low-molecular weight polymers. In various embodiments, the molecular weight of the polymer can be a wide range of values. The average molecular weight of the polymer can be from about 1000 to about 10,000,000; or about 1,000 to about 1,000,000; or about 5,000 to about 500,000; or about 10,000 to about 100,000; or about 20,000 to 50,000.

When the gel is designed to be a flowable gel, it can vary from low viscosity, similar to that of water, to high viscosity, similar to that of a paste, depending on the molecular weight and concentration of the polymer used in the gel. The viscosity of the gel can be varied such that the polymeric composition can be applied to a patient's tissues by any convenient technique, for example, by brushing, dripping, injecting, or painting. Different viscosities of the gel will depend on the technique used to apply the composition.

In various embodiments, the gel has an inherent viscosity (abbreviated as “I.V.” and units are in deciliters/gram), which is a measure of the gel's molecular weight and degradation time (e.g., a gel with a high inherent viscosity has a higher molecular weight and longer degradation time). Typically, a gel with a high molecular weight provides a stronger matrix and the matrix takes more time to degrade. In contrast, a gel with a low molecular weight degrades more quickly and provides a softer matrix. In various embodiments, the gel has a molecular weight, as shown by the inherent viscosity, from about 0.10 dL/g to about 1.2 dL/g or from about 0.10 dL/g to about 0.40 dL/g. Other IV ranges include but are not limited to about 0.05 to about 0.15 dL/g, about 0.10 to about 0.20 dL/g, about 0.15 to about 0.25 dL/g, about 0.20 to about 0.30 dL/g, about 0.25 to about 0.35 dL/g, about 0.30 to about 0.35 dL/g, about 0.35 to about 0.45 dL/g, about 0.40 to about 0.45 dL/g, about 0.45 to about 0.50 dL/g, about 0.50 to about 0.70 dL/g, about 0.60 to about 0.80 dL/g, about 0.70 to about 0.90 dL/g, and about 0.80 to about 1.00 dL/g.

In various embodiments, the gel can have a viscosity of about 300 to about 5,000 centipoise (cp). In other embodiments, the gel can have a viscosity of from about 5 to about 300 cps, from about 10 cps to about 50 cps, or from about 15 cps to about 75 cps at room temperature. The gel may optionally have a viscosity enhancing agent such as, for example, hydroxypropyl cellulose, hydroxypropyl methylcellulose, hydroxyethyl methylcellulose, carboxymethylcellulose and salts thereof, Carbopol, poly-(hydroxyethylmethacrylate), poly-(methoxyethylmethacrylate), poly(methoxyethoxyethyl methacrylate), polymethylmethacrylate (PMMA), methylmethacrylate (MMA), gelatin, polyvinyl alcohols, propylene glycol, mPEG, PEG 200, PEG 300, PEG 400, PEG 500, PEG 600, PEG 700, PEG 800, PEG 900, PEG 1000, PEG 1450, PEG 3350, PEG 4500, PEG 8000 or combinations thereof.

In various embodiments, the gel is a hydrogel made of high molecular weight biocompatible elastomeric polymers of synthetic or natural origin. A desirable property for the hydrogel to have is the ability to respond rapidly to mechanical stresses, particularly shears and loads, in the human body.

Hydrogels obtained from natural sources are particularly appealing because they are more likely to be biodegradable and biocompatible for in vivo applications. Suitable hydrogels include natural hydrogels, such as for example, gelatin, collagen, silk, elastin, fibrin and polysaccharide-derived polymers like agarose, and chitosan, glucomannan gel, hyaluronic acid, polysaccharides, such as cross-linked carboxyl-containing polysaccharides, or a combination thereof. Synthetic hydrogels include, but are not limited to those formed from polyvinyl alcohol, acrylamides such as polyacrylic acid and poly(acrylonitrile-acrylic acid), polyurethanes, polyethylene glycol (e.g., PEG 3350, PEG 4500, PEG 8000), silicone, polyolefins such as polyisobutylene and polyisoprene, copolymers of silicone and polyurethane, neoprene, nitrile, vulcanized rubber, poly(N-vinyl-2-pyrrolidone), acrylates such as poly(2-hydroxy ethyl methacrylate) and copolymers of acrylates with N-vinyl pyrolidone, N-vinyl lactams, polyacrylonitrile or combinations thereof. The hydrogel materials may further be cross-linked to provide further strength as needed. Examples of different types of polyurethanes include thermoplastic or thermoset polyurethanes, aliphatic or aromatic polyurethanes, polyetherurethane, polycarbonate-urethane or silicone polyether-urethane, or a combination thereof.

In various embodiments, rather than directly admixing the therapeutic agent into the gel, microspheres may be dispersed within the gel, the microspheres being loaded with clonidine, sulindac, and/or fluocinolone. In one embodiment, the microspheres provide for a sustained release of the clonidine, sulindac, and/or fluocinolone. In yet another embodiment, the gel, which is biodegradable, prevents the microspheres from releasing the clonidine, sulindac, and/or fluocinolone; the microspheres thus do not release the clonidine, sulindac, and/or fluocinolone until they have been released from the gel. For example, a gel may be deployed around a target tissue site (e.g., alveolar ridge). Dispersed within the gel may be a plurality of microspheres that encapsulate the desired therapeutic agent. Certain of these microspheres degrade once released from the gel, thus releasing the clonidine, sulindac, and/or fluocinolone.

Microspheres, much like a fluid, may disperse relatively quickly, depending upon the surrounding tissue type, and hence disperse the clonidine, sulindac, and/or fluocinolone. In some situations, this may be desirable; in others, it may be more desirable to keep the clonidine, sulindac, and/or fluocinolone tightly constrained to a well-defined target site. The present invention also contemplates the use of adherent gels to so constrain dispersal of the therapeutic agent. These gels may be deployed, for example, in the oral cavity, tooth, bone or in surrounding tissue.

Drug Delivery

It will be appreciated by those with skill in the art that the depot can be administered to the target site using a “cannula” or “needle” that can be a part of a drug delivery device e.g., a syringe, a gun drug delivery device, or any medical/dental device suitable for the application of a drug to the oral cavity or surrounding region. The cannula or needle of the drug depot device is designed to cause minimal physical and psychological trauma to the patient.

Cannulas or needles include tubes that may be made from materials, such as for example, polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof. The cannula or needle may optionally include one or more tapered regions. In various embodiments, the cannula or needle may be beveled. The cannula or needle may also have a tip style vital for accurate treatment of the patient depending on the site for implantation. In various embodiments, the cannula or needle may also be non-coring and have a sheath covering it to avoid unwanted needle sticks.

The dimensions of the hollow cannula or needle, among other things, will depend on the site for implantation. Some examples of lengths of the cannula or needle may include, but are not limited to, from about 50 to 150 mm in length, for example, about 65 mm for epidural pediatric use, about 85 mm for a standard adult and about 110 mm for an obese adult patient. The thickness of the cannula or needle will also depend on the site of implantation. In various embodiments, the thickness includes, but is not limited to, from about 0.05 to about 1.655 (mm). The gauge of the cannula or needle may be the widest or smallest diameter or a diameter in between for insertion into a human or animal body. The widest diameter is typically about 14 gauge, while the smallest diameter is about 25 gauge. In various embodiments the gauge of the needle or cannula is about 18 to about 25 gauge.

In various embodiments, like the drug depot and/or gel, the cannula or needle includes dose radiographic markers that indicate location at or near the target tissue site, so that the user may accurately position the depot at or near the site using any of the numerous diagnostic imaging procedures. Such diagnostic imaging procedures include, for example, X-ray imaging. Examples of such radiographic markers include, but are not limited to, barium, calcium, and/or metal beads or particles.

In various embodiments, the needle or cannula may include a transparent or translucent portion that can be visualizable by ultrasound, X-ray, or other imaging techniques. In such embodiments, the transparent or translucent portion may include a radiopaque material or ultrasound responsive topography that increases the contrast of the needle or cannula relative to the absence of the material or topography.

The drug depot, and/or medical/dental device to administer the drug may be sterilizable. In various embodiments, one or more components of the drug depot, and/or medical/dental device to administer the drug are sterilized by radiation in a terminal sterilization step in the final packaging. Terminal sterilization of a product provides greater assurance of sterility than from processes such as an aseptic process, which require individual product components to be sterilized separately and the final package assembled in a sterile environment.

Typically, in various embodiments, gamma radiation is used in the terminal sterilization step, which involves utilizing ionizing energy from gamma rays that penetrates deeply in the device. Gamma rays are highly effective in killing microorganisms, they leave no residues nor have sufficient energy to impart radioactivity to the device. Gamma rays can be employed when the device is in the package and gamma sterilization does not require high pressures or vacuum conditions, thus, package seals and other components are not stressed. In addition, gamma radiation eliminates the need for permeable packaging materials.

In various embodiments, electron beam (e-beam) radiation may be used to sterilize one or more components of the device. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity. E-beam sterilization may be used, for example, when the drug depot is included in a gel.

Other methods may also be used to sterilize the depot and/or one or more components of the device, including, but not limited to, gas sterilization, such as, for example, with ethylene oxide or steam sterilization.

In various embodiments, a kit is provided that may include additional parts along with the drug depot and/or medical/dental device combined together to be used to implant the drug depot. The kit may include the drug depot device in a first compartment. The second compartment may include a canister holding the drug depot and any other instruments needed for the localized drug delivery. A third compartment may include gloves, drapes, wound dressings and other procedural supplies for maintaining sterility of the implanting process, as well as an instruction booklet. A fourth compartment may include additional cannulas and/or needles. A fifth compartment may include an agent for radiographic imaging. Each tool may be separately packaged in a plastic pouch that is radiation sterilized. A cover of the kit may include illustrations of the implanting procedure and a clear plastic cover may be placed over the compartments to maintain sterility.

In various embodiments, a method is provided for treating periodontal disease in a patient in need of such treatment, the method comprises implanting into an oral cavity of the patient a drug depot comprising clonidine in an amount of from about 1 wt. % to about 20 wt. % of the drug depot, fluocinolone in an amount from about 0.05 wt. % to about 25 wt. % of the drug depot, and/or sulindac in an amount from about 20 wt. % to about 40 wt. % of the drug depot and at least one biodegradable polymer, wherein the drug depot is capable of releasing clonidine, fluocinolone and/or sulindac over a period of at least two weeks, thereby treating the periodontal disease.

In various embodiments, to reduce, prevent and/or treat periodontal disease, an opening is made at the implant site in the oral cavity (e.g., gum, tooth, bone, muscle, etc.). The opening can be made with, for example, a needle, cannula, scalpel, punch, plier, or other medical or dental instrument. The drug depot is inserted through the tissue down to the target tissue site and implanted at or near the target site.

In various embodiments, when the target site comprises a bone or tissue under the gum in the oral cavity, a portion of fluid (e.g., blood, etc.) can be withdrawn from the target site through the cannula or needle first and then the depot administered (e.g., placed, dripped, injected, or implanted, etc.). The target site will re-hydrate (e.g., replenishment of fluid) and this aqueous environment will cause the drug to be released from the depot.

In some embodiments, the drug depot comprises a pellet of 0.5 mm to 5 mm comprising a therapeutically effective amount of clonidine, sulindac, and/or fluocinolone and the periodontal treatment involves placing 1 to 6 drug depot pellets at target tissue sites in the oral cavity (e.g., gum, tooth, bone, muscle, near the periodontal pocket etc.), where the drug depot pellets release effective amounts of the drug(s) over a period of at least two weeks to two months.

When the drug depot comprises clonidine, in some embodiments, it is preferable to co-administer clonidine with an antagonist to counteract undesirable effects, for example the blood pressure decrease that can be caused by clonidine. Exemplary antagonists include but are not limited to phentolamine, yohimbine, tolazoline and piperoxane. Additionally, compounds such as 5-fluorodeoxyuridine (FUDR) and 3,4 dehydroprolene may also be included.

The clonidine, sulindac, and/or fluocinolone-based formulation of the present application may be used as medicaments in the form of pharmaceutical preparations. The preparations may be formed in a suitable pharmaceutical carrier that may be solid or liquid and organic or inorganic, and placed in the appropriate form for parenteral, local, or other administration as desired. As persons of ordinary skill are aware, known carriers include but are not limited to water, gelatin, lactose, starches, stearic acid, magnesium stearate, sicaryl alcohol, talc, vegetable oils, benzyl alcohols, gums, waxes, propylene glycol, polyalkylene glycols or other known carriers for medicaments.

Another embodiment is directed to a method for treating periodontal disease in a patient in need of such treatment, the method comprises implanting into an oral cavity of the patient a drug depot comprising clonidine in an amount of from about 1 wt. % to about 20 wt. % of the drug depot, fluocinolone in an amount from about 0.05 wt. % to about 25 wt. % of the drug depot, and/or sulindac in an amount from about 20 wt. % to about 40 wt. % of the drug depot and at least one biodegradable polymer, wherein the drug depot is capable of releasing clonidine, fluocinolone and/or sulindac over a period of at least two weeks, thereby treating the periodontal disease.

In some embodiments, the clonidine, fluocinolone and/or sulindac is encapsulated in a plurality of depots comprising microparticles, microspheres, microcapsules, and/or microfibers.

In some embodiments, the clonidine, fluocinolone and/or sulindac is suitable for parenteral administration. The term “parenteral” as used herein refers to modes of administration that bypass the gastrointestinal tract, and include for example, intravenous, intramuscular, continuous or intermittent infusion, intraperitoneal, intrasternal, subcutaneous, intra-operatively, intraarticular injection or combinations thereof. An injection may also be into a muscle, nerve, bone, gum or other tissue.

### Methods of Making Depots

In various embodiments, the drug depot comprising the clonidine, fluocinolone and/or sulindac can be made by combining a biocompatible polymer and a therapeutically effective amount of clonidine, fluocinolone and/or sulindac or pharmaceutically acceptable salt thereof and forming the implantable drug depot from the combination.

Various techniques are available for forming at least a portion of a drug depot from the biocompatible polymer(s), therapeutic agent(s), and optional materials, including solution processing techniques and/or thermoplastic processing techniques. Where solution processing techniques are used, a solvent system is typically selected that contains one or more solvent species. The solvent system is generally a good solvent for at least one component of interest, for example, biocompatible polymer and/or therapeutic agent. The particular solvent species that make up the solvent system can also be selected based on other characteristics, including drying rate and surface tension.

Solution processing techniques include solvent casting techniques, spin coating techniques, web coating techniques, solvent spraying techniques, dipping techniques, techniques involving coating via mechanical suspension, including air suspension (e.g., fluidized coating), ink jet techniques and electrostatic techniques. Where appropriate, techniques such as those listed above can be repeated or combined to build up the depot to obtain the desired release rate and desired thickness.

In various embodiments, a solution containing solvent and biocompatible polymer are combined and placed in a mold of the desired size and shape. In this way, polymeric regions, including barrier layers, lubricious layers, and so forth can be formed. If desired, the solution can further comprise, one or more of the following: clonidine and other therapeutic agent(s) and other optional additives such as radiographic agent(s), etc. in dissolved or dispersed form. This results in a polymeric matrix region containing these species after solvent removal. In other embodiments, a solution containing solvent with dissolved or dispersed therapeutic agent is applied to a pre-existing polymeric region, which can be formed using a variety of techniques including solution processing and thermoplastic processing techniques, whereupon the therapeutic agent is imbibed into the polymeric region.

Thermoplastic processing techniques for forming the depot or portions thereof include molding techniques (for example, injection molding, rotational molding, and so forth), extrusion techniques (for example, extrusion, co-extrusion, multi-layer extrusion, and so forth) and casting.

Thermoplastic processing in accordance with various embodiments comprises mixing or compounding, in one or more stages, the biocompatible polymer(s) and one or more of the following: clonidine, fluocinolone and/or sulindac optional additional therapeutic agent(s), radiographic agent(s), and so forth. The resulting mixture is then shaped into an implantable drug depot. The mixing and shaping operations may be performed using any of the conventional devices known in the art for such purposes.

During thermoplastic processing, there exists the potential for the therapeutic agent(s) to degrade, for example, due to elevated temperatures and/or mechanical shear that are associated with such processing. For example, clonidine, fluocinolone and/or sulindac may undergo substantial degradation under ordinary thermoplastic processing conditions. Hence, processing is preferably performed under modified conditions, which prevent the substantial degradation of the therapeutic agent(s). Although it is understood that some degradation may be unavoidable during thermoplastic processing, degradation is generally limited to 10% or less. Among the processing conditions that may be controlled during processing to avoid substantial degradation of the therapeutic agent(s) are temperature, applied shear rate, applied shear stress, residence time of the mixture containing the therapeutic agent, and the technique by which the polymeric material and the therapeutic agent(s) are mixed.

Mixing or compounding biocompatible polymer with therapeutic agent(s) and any additional additives to form a substantially homogenous mixture thereof may be performed with any device known in the art and conventionally used for mixing polymeric materials with additives.

Where thermoplastic materials are employed, a polymer melt may be formed by heating the biocompatible polymer, which can be mixed with various additives (e.g., therapeutic agent(s), inactive ingredients, etc.) to form a mixture. A common way of doing so is to apply mechanical shear to a mixture of the biocompatible polymer(s) and additive(s). Devices in which the biocompatible polymer(s) and additive(s) may be mixed in this fashion include devices such as single screw extruders, twin screw extruders, banbury mixers, high-speed mixers, ross kettles, and so forth.

Any of the biocompatible polymer(s) and various additives may be premixed prior to a final thermoplastic mixing and shaping process, if desired (e.g., to prevent substantial degradation of the therapeutic agent among other reasons).

For example, in various embodiments, a biocompatible polymer is precompounded with a radiographic agent (e.g., radio-opacifying agent) under conditions of temperature and mechanical shear that would result in substantial degradation of the therapeutic agent, if it were present. This precompounded material is then mixed with therapeutic agent under conditions of lower temperature and mechanical shear, and the resulting mixture is shaped into the clonidine containing drug depot. Conversely, in another embodiment, the biocompatible polymer can be precompounded with the therapeutic agent under conditions of reduced temperature and mechanical shear. This precompounded material is then mixed with, for example, a radio-opacifying agent, also under conditions of reduced temperature and mechanical shear, and the resulting mixture is shaped into the drug depot.

The conditions used to achieve a mixture of the biocompatible polymer and therapeutic agent and other additives will depend on a number of factors including, for example, the specific biocompatible polymer(s) and additive(s) used, as well as the type of mixing device used.

As an example, different biocompatible polymers will typically soften to facilitate mixing at different temperatures. For instance, where a depot is formed comprising PLGA or PLA polymer, a radio-opacifying agent (e.g., bismuth subcarbonate), and a therapeutic agent prone to degradation by heat and/or mechanical shear (e.g., clonidine), in various embodiments, the PGLA or PLA can be premixed with the radio-opacifying agent at temperatures of about, for example, 150° C. to 170° C. The therapeutic agent is then combined with the premixed composition and subjected to further thermoplastic processing at conditions of temperature and mechanical shear that are substantially lower than is typical for PGLA or PLA compositions. For example, where extruders are used, barrel temperature, volumetric output are typically controlled to limit the shear and therefore to prevent substantial degradation of the therapeutic agent(s). For instance, the therapeutic agent and premixed composition can be mixed/compounded using a twin screw extruder at substantially lower temperatures (e.g., 100-105° C.), and using substantially reduced volumetric output (e.g., less than 30% of full capacity, which generally corresponds to a volumetric output of less than 200 cc/min). It is noted that this processing temperature is well below the melting points of clonidine because processing at or above these temperatures will result in substantial therapeutic agent degradation. It is further noted that in certain embodiments, the processing temperature will be below the melting point of all bioactive compounds within the composition, including the therapeutic agent. After compounding, the resulting depot is shaped into the desired form, also under conditions of reduced temperature and shear.

In other embodiments, biodegradable polymer(s) and one or more therapeutic agents are premixed using non-thermoplastic techniques. For example, the biocompatible polymer can be dissolved in a solvent system containing one or more solvent species. Any desired agents (for example, a radio-opacifying agent, a therapeutic agent, or both radio-opacifying agent and therapeutic agent) can also be dissolved or dispersed in the solvents system. Solvent is then removed from the resulting solution/dispersion, forming a solid material. The resulting solid material can then be granulated for further thermoplastic processing (for example, extrusion) if desired.

As another example, the therapeutic agent can be dissolved or dispersed in a solvent system, which is then applied to a pre-existing drug depot (the pre-existing drug depot can be formed using a variety of techniques including solution and thermoplastic processing techniques, and it can comprise a variety of additives including a radio-opacifying agent and/or viscosity enhancing agent), whereupon the therapeutic agent is embedded on or in the drug depot. As above, the resulting solid material can then be granulated for further processing, if desired.

Typically, an extrusion process may be used to form the drug depot comprising a biocompatible polymer(s), therapeutic agent(s) and radio-opacifying agent(s). Co-extrusion may also be employed, which is a shaping process that can be used to produce a drug depot comprising the same or different layers or regions (for example, a structure comprising one or more polymeric matrix layers or regions that have permeability to fluids to allow immediate and/or sustained drug release). Multi-region depots can also be formed by other processing and shaping techniques such as co-injection or sequential injection molding technology.

In various embodiments, the depot that may emerge from the thermoplastic processing (e.g., pellet) is cooled. Examples of cooling processes include air cooling and/or immersion in a cooling bath. In some embodiments, a water bath is used to cool the extruded depot. However, where a water-soluble therapeutic agent such as clonidine, fluocinolone and/or sulindac is used, the immersion time should be held to a minimum to avoid unnecessary loss of therapeutic agent into the bath.

In various embodiments, immediate removal of water or moisture by use of ambient or warm air jets after exiting the bath will also prevent re-crystallization of the drug on the depot surface, thus controlling or minimizing a high drug dose “initial burst” or “bolus dose” upon implantation or insertion if this is release profile is not desired.

In various embodiments, the drug depot can be prepared by mixing or spraying the drug with the polymer and then molding the depot to the desired shape. In various embodiments, clonidine is used and mixed or sprayed with the PLGA or PEG550 polymer, and the resulting depot may be formed by extrusion and dried.

In various embodiments, there is a pharmaceutical formulation comprising: clonidine, fluocinolone and/or sulindac, wherein the clonidine comprises from about 5 wt. % to about 15 wt. % of the formulation, the fluocinolone comprises from about 1 wt. % to about 15 wt. % of the formulation, and/or the sulindac comprises from about 5 wt. % to about 15 wt. % of the formulation. By way of example, when using a 5%-15% clonidine composition, the mole ratio of clonidine to polymer would be from approximately 16-52 when using an approximately 80 kDalton polymer that has a 267 grams/mole ratio.

In some embodiments, the at least one biodegradable polymer comprises poly(lactic-co-glycolide) (PLGA) or poly(orthoester) (POE) or a combination thereof. The poly(lactic-co-glycolide) may comprise a mixture of polyglycolide (PGA) and polylactide and in some embodiments, in the mixture, there is more polylactide than polyglycolide. In various embodiments there is 100% polylactide and 0% polyglycolide; 95% polylactide and 5% polyglycolide; 90% polylactide and 10% polyglycolide; 85% polylactide and 15% polyglycolide; 80% polylactide and 20% polyglycolide; 75% polylactide and 25% polyglycolide; 70% polylactide and 30% polyglycolide; 65% polylactide and 35% polyglycolide; 60% polylactide and 40% polyglycolide; 55% polylactide and 45% polyglycolide; 50% polylactide and 50% polyglycolide; 45% polylactide and 55% polyglycolide; 40% polylactide and 60% polyglycolide; 35% polylactide and 65% polyglycolide; 30% polylactide and 70% polyglycolide; 25% polylactide and 75% polyglycolide; 20% polylactide and 80% polyglycolide; 15% polylactide and 85% polyglycolide; 10% polylactide and 90% polyglycolide; 5% polylactide and 95% polyglycolide; and 0% polylactide and 100% polyglycolide.

In various embodiments that comprise both polylactide and polyglycolide; there is at least 95% polylactide; at least 90% polylactide; at least 85% polylactide; at least 80% polylactide; at least 75% polylactide; at least 70% polylactide; at least 65% polylactide; at least 60% polylactide; at least 55%; at least 50% polylactide; at least 45% polylactide; at least 40% polylactide; at least 35% polylactide; at least 30% polylactide; at least 25% polylactide; at least 20% polylactide; at least 15% polylactide; at least 10% polylactide; or at least 5% polylactide; and the remainder of the biopolymer is polyglycolide.

In various embodiments, the drug particle size is from about 5 to 30 micrometers, however, in various embodiments ranges from about 1 micron to 250 microns may be used. In some embodiments, the biodegradable polymer comprises at least 50 wt. %, at least 60 wt. %, at least 70 wt. %, at least 80 wt. % of the formulation, at least 85 wt. % of the formulation, at least 90 wt. % of the formulation, at least 95 wt. % of the formulation, at least 97 wt. % of the formulation, at least 99 wt. % of the formulation. In some embodiments, the at least one biodegradable polymer and the clonidine, sulindac, and/or fluocinolone are the only components of the pharmaceutical formulation.

In some embodiments, at least 75% of the particles have a size from about 10 micrometer to about 200 micrometers. In some embodiments, at least 85% of the particles have a size from about 10 micrometer to about 200 micrometers. In some embodiments, at least 95% of the particles have a size from about 10 micrometer to about 200 micrometers. In some embodiments, all of the particles have a size from about 10 micrometer to about 200 micrometers.

In some embodiments, at least 75% of the particles have a size from about 20 micrometer to about 180 micrometers. In some embodiments, at least 85% of the particles have a size from about 20 micrometers to about 180 micrometers. In some embodiments, at least 95% of the particles have a size from about 20 micrometer to about 180 micrometers. In some embodiments, all of the particles have a size from about 20 micrometer to about 180 micrometers.

In some embodiments, there is a pharmaceutical formulation comprising: clonidine, wherein the clonidine is in the form of a hydrochloride salt, and comprises from about 1 wt. % to about 20 wt. % of the formulation, the sulindac comprises sulindac sodium from about 5 wt. % to about 15 wt. % of the drug depot; and/or the fluocinolone comprises fluocinolone acetonide from about 1 wt. % to about 15 wt. % of the drug depot, and at least one biodegradable polymer, wherein the at least one biodegradable polymer comprises poly(lactide-co-glycolide) (or poly(lactic-co-glycolic acid)) or poly(orthoester) or a combination thereof, and said at least one biodegradable polymer comprises at least 80 wt. % to 90 wt % of said formulation.

In some embodiments, there are methods for treating periodontal disease. These methods comprise: administering a pharmaceutical composition to an organism, wherein said pharmaceutical composition comprises from about 0.05 wt. % to about 20 wt. % of the formulation, and at least one biodegradable polymer. In some embodiments, the loading is from about 5 wt. % to about 15 wt. %. In some embodiments, the loading is from about 10 wt. % to about 20 wt. %.

In some embodiment there is a higher loading of clonidine, sulindac, and/or fluocinolone, e.g., at least 20 wt. %, at least 30 wt. %, at least 40 wt. %, at least 50 wt. %, at least 60 wt. %, at least 70 wt. %, at least 80 wt. %, or at least 90 wt. %.

A strategy of triangulation may be effective when administering these pharmaceutical formulations. Thus, a plurality (at least two, at least three, at least four, at least five, at least six, at least seven, etc.) drug depots comprising the pharmaceutical formulations may be placed around the target tissue site (also known as the area of periodontal disease) such that the target tissue site falls within a region that is either between the formulations when there are two, or within an area whose perimeter is defined by a set of plurality of formulations.

In some embodiments, the formulations are slightly rigid with varying length, widths, diameters, etc. For example, certain formulations may have a diameter of 0.50 mm and a length of 4 mm. It should be noted that particle size may be altered by techniques such as mort and pestel, jet-drying or jet milling.

In some embodiments, clonidine is released at a rate of 2-3 μg per day for a period of at least 3 days. In some embodiments, this release rate continues for, at least ten days, at least fourteen days, at least twenty-one days, at least thirty days, at least sixty days, at least one hundred days, at least one-hundred and thirty-five days, at least one-hundred and fifty days, or at least one hundred and eighty days. For some embodiments, 300-425 micrograms of clonidine as formulated with a biopolymer are implanted into a person at or near a target tissue site. If clonidine is implanted at multiple sites that triangulate the target site then in some embodiments, the total amount of clonidine at each site is a fraction of the total 300-425 micrograms. For example, one may implant a single does of 324 micrograms at one site, or two separate doses of 162 micrograms at two sites, or three separate dose of 108 micrograms at three sites that triangulate the tissue site. It is important to limit the total dosage to an amount less than that which would be harmful to the organism. However, in some embodiments, although when there are a plurality of sites each site may contain less than the total dose that might have been administered in a single application, it is important to remember that each site will independent have a release profile, and the biopolymers' concentration and substance should be adjusted accordingly to ensure that the sustain release occurs over sufficient time. Of course the duration of treatment will depend on the severity of periodontal disease. Treatments for periodontal disease, typically can be from two weeks to two months.

The dosage may be from approximately 0.0005 to approximately 960 μg/day. Additional dosages of clonidine include from approximately 0.0005 to approximately 900 μg/day; approximately 0.0005 to approximately 500 μg/day; approximately 0.0005 to approximately 250 μg/day; approximately 0.0005 to approximately 100 μg/day; approximately 0.0005 to approximately 75 μg/day; approximately 0.001 to approximately 70 μg/day; approximately 0.001 to approximately 65 μg/day; approximately 0.001 to approximately 60 μg/day; approximately 0.001 to approximately 55 μg/day; approximately 0.001 to approximately 50 μg/day; approximately 0.001 to approximately 45 μg/day; approximately 0.001 to approximately 40 μg/day; approximately 0.001 to approximately 35 μg/day; approximately 0.0025 to approximately 30 μg/day; approximately 0.0025 to approximately 25 μg/day; approximately 0.0025 to approximately 20 μg/day; approximately 0.0025 to approximately 15 μg/day; approximately 0.0025 to approximately 10 μg/day; approximately 0.0025 to approximately 5 μg/day; and approximately 0.0025 to approximately 2.5 μg/day. In another embodiment, the dosage of clonidine is from approximately 0.005 to approximately 15 μg/day. In another embodiment, the dosage of clonidine is from approximately 0.005 to approximately 10 μg/day. In another embodiment, the dosage of clonidine is from approximately 0.005 to approximately 5 μg/day. In another embodiment, the dosage of clonidine is from approximately 0.005 to 2.5 μg/day. In some embodiments, the amount of clonidine is between 40 and 600 μg/day. In some embodiments, the amount of clonidine is between 200 and 400 μg/day.

In one exemplary dosing regiment, a rat may be provided with sufficient clonidine in a biodegradable polymer to provide sustain release of 0.240 μg/day for 135 days. The total amount of clonidine that is administered over this time period would be approximately 32.4 μg. In another exemplary dosing regimen, a human is provided with sufficient clonidine in a biodegradable polymer to provide sustain release of 2.4 μg/day for 135 days. The total amount of clonidine that is administered over this time period would be approximately 324 μg.

When using a plurality of pellets, the pellet number is based on the amount of drug loading into a pellet of appropriate size (i.e., 0.5 mm diameter×4 mm length) and how much drug is needed (e.g., approximately 325 μg clonidine (3 pellets)). In some embodiments there is a polymer that releases a bolus amount of compound over the first few (˜5) days before it settles down and releases 2.5 mg/day for 135 days. An exemplary formulation is 5% wt. to 8% wt. clonidine, 100 DL 5E.

In some embodiments, the polymer depots enable one to provide efficacy of the active ingredient that is equivalent to clonidine subcutaneous injections that deliver more than 2.5 times as much drug.

In some embodiments, when the drug depot comprises fluocinolone, the dosage of fluocinolone may be from approximately 0.0005 to approximately 100 μg/day. Additional dosages of fluocinolone include from approximately 0.0005 to approximately 50 μg/day; approximately 0.0005 to approximately 25 μg/day; approximately 0.0005 to approximately 10 μg/day; approximately 0.0005 to approximately 5 μg/day; approximately 0.0005 to approximately 1 μg/day; approximately 0.005 to approximately 0.75 μg/day; approximately 0.0005 to approximately 0.5 μg/day; approximately 0.0005 to approximately 0.25 μg/day; approximately 0.0005 to approximately 0.1 μg/day; approximately 0.0005 to approximately 0.075 μg/day; approximately 0.0005 to approximately 0.05 μg/day; approximately 0.001 to approximately 0.025 μg/day; approximately 0.001 to approximately 0.01 μg/day; approximately 0.001 to approximately 0.0075 μg/day; approximately 0.001 to approximately 0.005 μg/day; approximately 0.001 to approximately 0.025 μg/day; and 0.002 to approximately 0.025 μg/day. In another embodiment, the dosage of fluocinolone is from approximately 0.001 to approximately 15 μg/day. In another embodiment, the dosage of fluocinolone is from approximately 0.001 to approximately 10 μg/day. In another embodiment, the dosage of fluocinolone is from approximately 0.001 to approximately 5 μg/day. In another embodiment, the dosage of fluocinolone is from approximately 0.001 to 2.5 μg/day. In some embodiments, the amount of fluocinolone is between 40 and 600 μg/day. In some embodiments, the amount of fluocinolone is between 200 and 400 μg/day. Dosing formulations may be prepared to contain a sufficient amount of the active ingredient to enable the desired about of compound to be release over the desired amount of time.

In some embodiments, there is sufficient fluocinolone such that the fluocinolone is released at a rate of 2-3 μg per day for a period of at least three days. In some embodiments, this release rate continues for, at least ten days, at least fourteen days, at least twenty-one days, at least thirty days, at least fifty days, at least sixty days, at least one hundred days, at least one-hundred and thirty-five days, at least one-hundred and fifty days, or at least one hundred and eighty days.

For some embodiments, 300-350 micrograms of fluocinolone as formulated with a biopolymer are implanted into a person at or near a target tissue site. If fluocinolone is implanted at multiple sites that triangulate or line the target site then in some embodiments, the total amount of fluocinolone at each site is a fraction of the total 300-350 micrograms. For example, one may implant a single does of 324 micrograms at one site, or two separate doses of 162 micrograms at two sites, or three separate dose of 108 micrograms at three sites that triangulate the tissue site. It is important to limit the total dosage to an amount less than that which would be harmful to the organism. However, in some embodiments, although when there are a plurality of sites each site may contain less than the total does that might have been administered in a single application, it is important to remember that each site will independent have a release profile, and the biopolymers' concentration and substance should be adjusted accordingly to ensure that the sustain release occurs over sufficient time.

In some embodiments, when the drug depot comprises sulindac, it is released at a rate of 5-15 mg/per day, or 7-12 mg/day or 8-10 mg/day for a period of at least two weeks to two months. In some embodiments, this release rate lasts for, at least thirty days, at least sixty days, at least one hundred days, at least one-hundred and thirty-five days, at least one-hundred and fifty days, or at least one hundred and eighty days.

For some embodiments, 625-2025 milligrams of sulindac as formulated with a biopolymer are implanted into a person at or near a target tissue site. For some embodiments, 945-1620 milligrams of sulindac as formulated with a biopolymer are implanted into a person at or near a target tissue site. For some embodiments, 1080-1325 milligrams of sulindac as formulated with a biopolymer are implanted into a person at or near a target tissue site.

If sulindac is implanted at multiple sites that triangulate the target site then in some embodiments, the total amount of sulindac at each site is a fraction of the total number of milligrams. For example, one may implant a single does of 1296 milligrams at one site, or two separate doses of 648 micrograms at two sites, or three separate dose of 432 milligrams at three sites that triangulate the tissue site. It is important to limit the total dosage to an amount less than that which would be harmful to the organism. However, in some embodiments, although when there are a plurality of sites each site may contain less than the total does that might have been administered in a single application, it is important to remember that each site will independent have a release profile, and the biopolymers' concentration and substance should be adjusted accordingly to ensure that the sustain release occurs over sufficient time.

In one embodiment of the present invention, the sulindac and the dosage is from approximately 0.001 μg/day to approximately 100 mg/day. Additional dosages of sulindac can include from approximately 0.001 μg/day to approximately 200 mg/day; approximately 0.001 μg/day to approximately 100 mg/day; approximately 0.001 μg/day to approximately 1 mg/day; approximately 0.001 μg/day to approximately 500 μg/day; approximately 0.001 μg/day to approximately 100 μg/day; approximately 0.025 to approximately 75 μg/day; approximately 0.025 μg/day to approximately 65 μg/day; approximately 0.025 μg/day to approximately 60 μg/day; approximately 0.025 μg/day to approximately 55 μg/day; approximately 0.025 μg/day to approximately 50 μg/day; approximately 0.025 μg/day to approximately 45 μg/day; approximately 0.025 μg/day to approximately 40 μg/day; approximately 0.025 μg/day to approximately 35 μg/day; approximately 0.005 to approximately 30 μg/day; approximately 0.005 to approximately 25 μg/day; approximately 0.005 μg/day to approximately 20 μg/day; and approximately 0.005 μg/day to approximately 15 μg/day. In another embodiment, the dosage of sulindac is from approximately 0.01 μg/day to approximately 15 μg/day. In another embodiment, the dosage of sulindac is from approximately 0.01 μg/day to approximately 10 μg/day. In another embodiment, the dosage of sulindac is from approximately 0.01 μg/day to approximately 5 μg/day. In another embodiment, the dosage of sulindac is from approximately 0.01 μg/day to approximately 20 μg/day. In another embodiment, sulindac is administered in a drug depot that releases 9.6 μg/day.

Having now generally described the invention, the same may be more readily understood through the following reference to the following examples, which are provided by way of illustration and are not intended to limit the present invention unless specified.

### EXAMPLES

### Example 1

The purpose of this set of experiments was to narrow the number of periodontal candidates by determining the lowest dose of the drug that inhibits or blocks osteoclast differentiation and/or resorption.

Methods:

### Osteoclast Differentiation and Activity—Cell Culture and Differentiation:

1. Cell culture complete media was prepared by adding 10% FBS to high glucose DMEM.
2. A nitric oxide-containing murine RAW NO+ cell line, f frozen in DMSO-containing medium under liquid nitrogen, was used for these assays.
3. Cells were thawed in a 37° C. water bath and resuspended in complete media (see #1 above).
4. The cells were plated in a 175 cm² polystyrene, white top, non-adherent culture flask at a density of 1×10⁴ cells/cm² and allowed to settle overnight (Day-1, Table G).
5. The media was changed approximately 24 hours after the initial plating (Day 0, Table G).
6. After approximately two days of proliferation, the cells were harvested with a cell scraper, collected in a 50 mL conical tube and centrifuged at 1000 rpm to pellet the cells.
7. The cell pellets were resuspended in 4 mL complete media, counted with a hemocytometer and plated at a density of 0.125×10⁴ cells/0.25 ml/well in a 16 well osteologic assay slide (Day 1, Table G).
8. The cells were allowed to adhere for 24 hours, the media was removed and fresh differentiation media was prepared that contained complete media+RANK L (30 ng/ml; Pepro Tech #315-11)+/−drug (Day 2, Table G). RANK is receptor activator or NF_{κ}β.
9. Step 8 was repeated on days 5 and 7 (Table G).
10. On day 9 the plates were harvested by removing the media, rinsing with dH₂O and treated with bleach for 7 minutes.
11. The bleach was disposed of in the drug waste container and the wells and gaskets were removed from the slide.
12. The plates were rinsed with H₂O 5 times, dried and analyzed for matrix degradation as described below

TABLE G
Calendar of how the differentiation
and activity experiments were ran

| Tuesday | Wednesday | Thursday | Friday | Sat/Sun |
|---|---|---|---|---|
| (Day 1) | (Day 0) | (Day 1) | (Day 2) | (Day 3/4) |
| Raw cells | Media | Cells passaged | Fed differen- | |
| thawed and | changed | and plated on | tiation | |
| plated | | osteologic 16 | medium + | |
| | | well slides | drug | |
| Monday | Tuesday | Wednesday | Thursday | Friday |
| (Day 5) | (Day 6) | (Day 7) | (Day 8) | (Day 9) |
| Fed differen- | | Fed differen- | | Harvested |
| tiation | | tiation | | |
| medium + | | medium + | | |
| drug | | drug | | |

The clonidine concentrations tested were 100 μM, 10 μM, 1 μM, 0.1 μM and the sulindac concentrations tested were 140 μM, 70 μM, 35 μM, 17.5 μM, and 8.75 μM. Clonidine was found to have significant inhibitory effects on osteoclast differentiation and resorption. The lowest inhibitory concentration of clonidine tested was 10 μM. All doses of clonidine allowed for monocyte/osteoclast cell proliferation that was similar to the control wells. This suggests that clonidine does not inhibit cell proliferation, but inhibits differentiation.

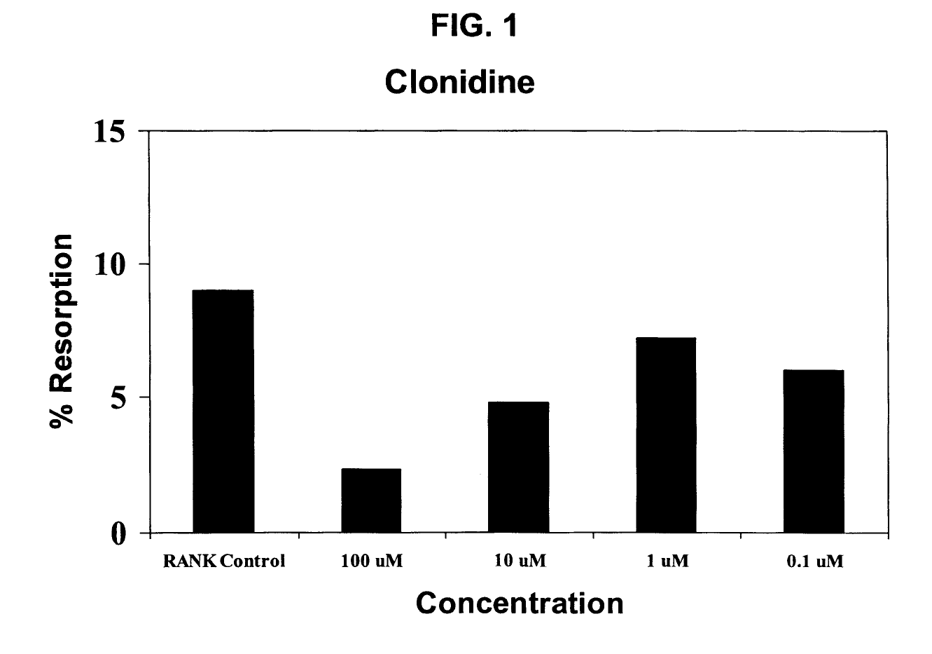

FIG. 1 illustrates the effect of clonidine on osteoclast differentiation and resorption. Clonidine significantly decreased osteoclast differentiation and resorption at 100 mM and 10 mM when compared to the RANK only treated control wells. This indicates that clonidine may be useful in reducing, preventing, and/or treating periodontal disease.

One of the more serious manifestations of periodontal disease is bone resorption. The inflammatory response the body mounts against the bacterial plaque releases factors that bring resorptive cells to the area. Those macrophage like cells convert to osteoclasts, the purpose of which are to remove diseased or old bone. This is an overreaction, such as often occurs in inflammation, and removes bone thereby loosening the teeth. The teeth may even be lost. In this study, clonidine has both been demonstrated to slow down bone resorption and to decrease inflammation, both of which would be very helpful to sufferers of the disease.

With regard to sulindac, this drug had significant inhibitory effects on osteoclast differentiation and resorption. The lowest inhibitory concentration of sulindac was 140.3 μM.

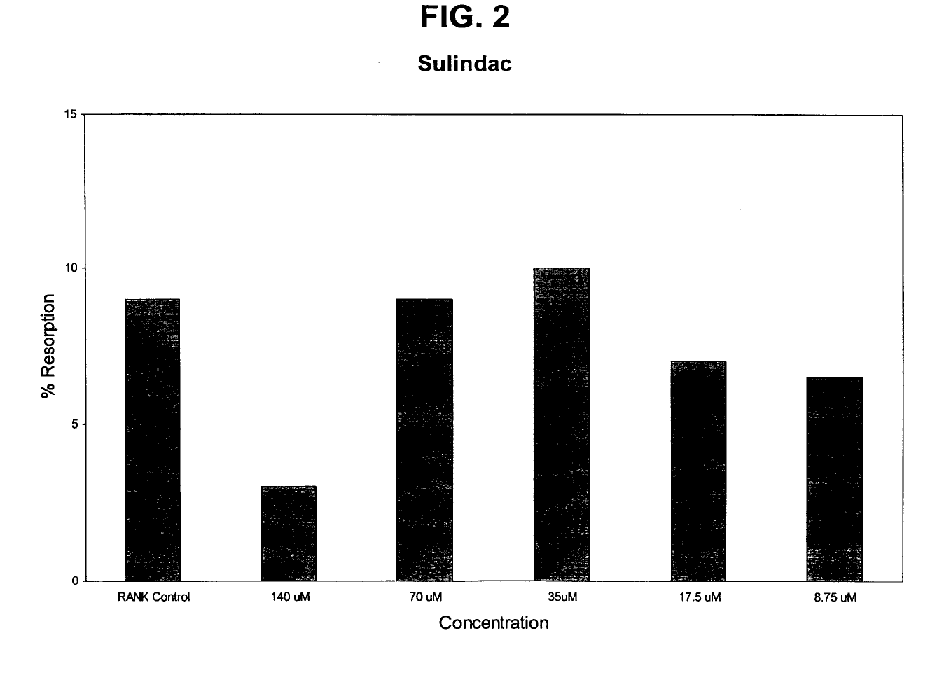

FIG. 2 illustrates the effect of sulindac on osteoclast differentiation and resorption Sulindac significantly decreased osteoclast differentiation at 140.3 μM when compared to the RANK only treated controls. The assay tested in Example 1 can be used to show the ability of selected drugs to inhibit osteoclast differentiation and resorption. The drugs that inhibit at the lowest concentrations and over a broad range of concentrations are preferred for polymer formulation and are most likely to have an effect on bone remodeling in vivo. In this study, sulindac decreased osteoclast differentiation and thus inflammation and also slowed down bone resorption, both of which is helpful in periodontal disease.

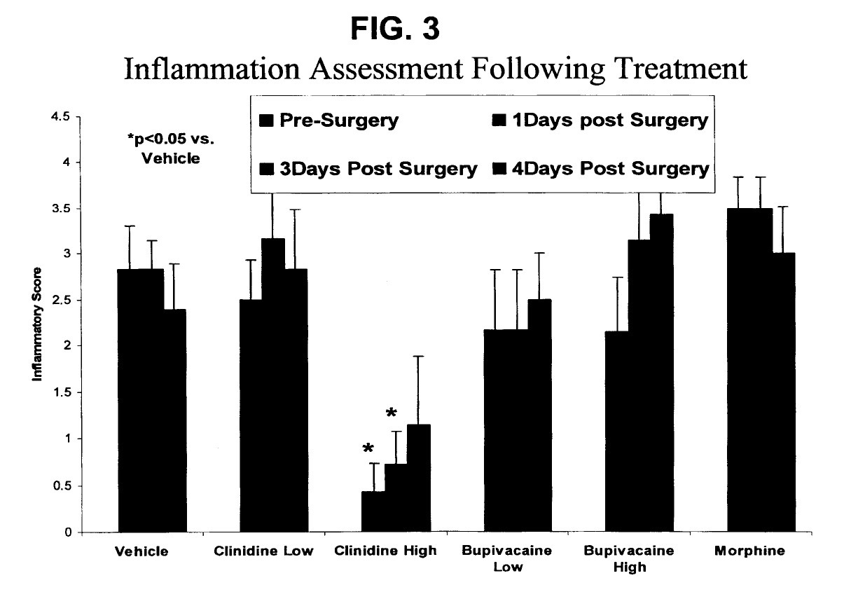

In a separate experiment, inflammation was induced in minipigs and the minipigs were given clonidine, bupivacaine, or morphine. The surgical site was reviewed for inflammation while the minipigs were anesthesized and on day 4. Scoring of inflammation was reviewed in the area of incision for redness with the following scores: 0—no redness; 1—slightly red limited to area of incision; 2—massive spread redness and swelling with the following scores 0—no swelling; 1—slightly swelling; 3—massive swelling. Redness and swelling were summed up so the minimum score is 0 and maximum score is 4. FIG. 3 is a graphic representation of inflammation assessment following treatment of the minipigs with injected drug on days 1, 3 and 4 post surgery. High dose clonidine (150 μg) had a very low inflammation score, which may be useful in treating periodontal disease. The low dose clonidine given was 75 μg.

Clonidine

The examples below show certain particularly advantageous results wherein the initial burst is not too large (i.e., not more than 7% of the load drug in the first five days) and the daily dose is approximately 2.4 μg/day±0.5 μg/day and can be released for 135 days. Also, clonidine drug loadings 5 wt. % to 8 wt. % provide advantageous results.

A 2-month chronic constriction injury (CCI) model of neuropathic pain was used to evaluate different formulations of a corticosteroid, fluocinolone, encapsulated in bioerodable polymers compared to fluocinolone given subcutaneously (SC). Different formulations as provided in Table 5 below were evaluated for reducing pain-associated behaviors: Thermal paw withdrawal latency was evaluated at baseline 7, 14, 21, 28, 35, 42, 49, 56 and 64 days post-operatively, while mechanical threshold was evaluated at 8, 15, 22, 29, 36, 43, 50, 57 and 64 days post-operatively.

The In-Vitro Elution Studies were carried out at 37° C. in phosphate-buffered saline (PBS, pH 7.4). Briefly, the rods (n=3) were weighed prior to immersion in 5 mL of PBS. At regular time intervals, the PBS was removed for analysis and replaced with 5 mL of fresh PBS. The PBS-elution buffer was analyzed for clonidine content using UV-Vis spectrometry.

### Example 2

### Formulation Testing

A number of clonidine formulations were prepared in which the polymer type, drug load, excipient (including some formulations in which there was no excipient), pellet size and processing were varied. These formulations are described below in Table 1, Table 2 and Table 3. A number of tests were performed on these formulations, including in vitro release tests in which the number of micrograms released was measured, as well as the cumulative percentage release of clonidine. Some of the formulation's elution profiles were tested and ranged from 10 days to 140 days with clonidine release rates of 1% to 100% cumulative release over 20, 40, 50, 80, and 140 days. These types of release profiles would be beneficial in reducing, treating or preventing periodontal disease, while decreasing pain.

The In-Vitro Elution Studies were carried out at 37° C. in phosphate-buffered saline (PBS, pH 7.4). The rods (n=3) were weighed prior to immersion in 5 mL of PBS. At regular time intervals, the PBS was removed for analysis and replaced with 5 mL of fresh PBS. The PBS-elution buffer was analyzed for clonidine content using UV-Vis spectrometry.

TABLE 1

| | | Drug | | Pellet Size (L × | |
|---|---|---|---|---|---|
| | | Load | | Dia; mm) or | |
| Notebook ID | Polymer Type | (Wt %) | Excipient | Description | Processing |
| 13335-60-1 | 8515 DLG 7E | 10 | N/A | 0.75 × 0.75 | Melt extrusion, co-spray dried |
| | | | | | drug/polymer |
| 13335-60-2 | 8515 DLG 7E | 10 | N/A | 0.75 × 0.75 | Melt extrusion, spray dried drug |
| 13335-60-3 | 8515 DLG 7E | 10 | N/A | 0.75 × 0.75 | Melt extrusion, hand ground drug |
| 13335-60-4 | 8515 DLG 7E | 10 | N/A | 0.75 × 0.75 | Melt extrusion, hand ground drug, |
| | | | | | spray dried polymer |
| 13335-60-5 | 8515 DLG 7E | 10 | N/A | 0.75 × 0.75 | Melt extrusion w/ recycle loop, hand |
| | | | | | ground drug |
| 13335-65-1 | 8515 DLG 7E | 5 | N/A | 3.0 × 0.75 | Melt extrusion, spray dried drug |
| 13335-65-2 | 8515 DLG 7E | 10 | N/A | 1.5 × 0.75 | Melt extrusion, spray dried drug |
| 13335-65-3 | 8515 DLG 7E | 20 | N/A | 0.75 × 0.75 | Melt extrusion, spray dried drug |
| 13335-65-4 | 100 DL 7E | 5 | N/A | 3.0 × 0.75 | Melt extrusion, spray dried drug |
| 13335-65-5 | 100 DL 7E | 10 | N/A | 1.5 × 0.75 | Melt extrusion, spray dried drug |
| 13335-65-6 | 100 DL 7E | 20 | N/A | 0.75 × 0.75 | Melt extrusion, spray dried drug |
| 13335-97-1 | 8515 DLG 7E | 7.5 | N/A | 3.0 × 0.75 | Melt extrusion, spray dried drug |
| 13335-97-2 | 100 DL 7E | 5 | N/A | 3.0 × 0.75 | Melt extrusion, spray dried drug |
| 13335-97-3 | 8515 DLG 7E | 5 | 10% mPEG | 3.0 × 0.75 | Melt extrusion, spray dried drug |
| 13335-97-4 | 100 DL 7E | 5 | 10% mPEG | 3.0 × 0.75 | Melt extrusion, spray dried drug |
| 13699-1-1 | 100 DL 7E | 5 | N/A | 3.0 × 0.75 | Melt extrusion, spray dried drug |
| 13699-16-1 | 8515 DLG 7E | 10 | N/A | 1.5 × 0.75 | Melt extrusion, spray dried drug |
| 13699-16-2 | 9010 DLG 7E | 10 | N/A | 1.5 × 0.75 | Melt extrusion, spray dried drug |
| 13699-16-3 | 9010 DLG 7E | 5 | N/A | 3.0 × 0.75 | Melt extrusion, spray dried drug |
| 13699-16-4 | 8515 DLG 7E | 5 | 5% mPEG | 3.0 × 0.75 | Melt extrusion, spray dried drug |
| 13699-16-5 | 8515 DLG 7E | 5 | 2.5% | 3.0 × 0.75 | Melt extrusion, spray dried drug |
| | | | mPEG | | |
| 13699-20-1 | 8515 DLG 7E | 5 | 1% MgO | 3.0 × 0.75 | Melt extrusion, spray dried drug |
| 13699-20-4 | 8515 DLG 7E | 5 | N/A | 3.0 × 0.75 | Melt extrusion, spray dried drug |
| 13699-20-5 | 100 DL 7E | 5 | 10% 5050 | 3.0 × 0.75 | Melt extrusion, spray dried drug |
| | | | DLG 6E | | |
| 13699-20-6 | 100 DL 7E | 5 | 10% 5050 | 3.0 × 0.75 | Melt extrusion, spray dried drug |
| | | | DLG 1A | | |
| 13699-20-7 | 8515 DLG | 10 | N/A | 1.5 × 0.75 | Melt extrusion, spray dried drug |
| | Purac | | | | |
| 13699-20-8 | 8515 DLG 7E | 5 | N/A | 3.0 × 0.75 | Melt extrusion 2X, spray dried drug |
| 13699-28-1 | 8515 DLG | 7.5 | N/A | 3.0 × 0.75 | Melt extrusion, spray dried drug |
| | Purac | | | | |
| 13699-28-2 | 8516 DLG | 12.5 | N/A | 2.0 × 0.75 | Melt extrusion, spray dried drug |
| | Purac | | | | |
| 13699-28-3 | 100 DL 7E | 5 | N/A | 3.0 × 0.75 | Melt extrusion, spray dried drug |
| 13699-31-1 | 8515 DLG 7E | 10 | N/A | N/A | heat press, spray dried drug |
| 13699-31-2 | 8515 DLG 7E | 10 | N/A | N/A | heat press, spray dried drug |
| 13699-31-3 | 8515 DLG 7E | 10 | N/A | N/A | heat press, spray dried drug |
| 13699-31-4 | 8515 DLG 7E | 10 | N/A | N/A | Melt extrusion, spray dried drug |
| 12702-13-4-a | 1,6- | 10 | N/A | 3 × 3 | Melt extrusion |
| | Hexanediol/ | | | | |
| | tCHDM | | | | |
| 12702-13-4-b | 75/25 PLGA | 10 | N/A | 3 × 3 | Melt extrusion |
| 12702-68-12 | 75/25 PLGA | 5 | mPEG | 1 × 1 | Melt extrusion |
| 12702-68-13 | 75/25 PLGA | 5 | TBO-Ac | 1 × 1 | Melt extrusion |
| 12702-72-1 | 75/25 PLGA | 5 | mPEG | 1 × 1 | Melt extrusion |
| 12702-80-7 | 75/25 PLGA | 10 | mPEG | 0.75 × 0.75 | Melt extrusion |
| 12702-80-8 | 75/25 PLGA | 15 | mPEG | 0.75 × 0.75 | Melt extrusion |
| 13395-3-1 | 85/15 PLGA | 10 | mPEG | 0.75 × 0.75 | Melt extrusion |
| 13395-3-2 | 85/15 PLGA | 15 | mPEG | 0.75 × 0.75 | Melt extrusion |
| 13395-3-3 | 85/15 PLGA | 5 | mPEG | 0.75 × 0.75 | Melt extrusion |
| 13395-15 | 85/15 PLGA | 15 | mPEG | 0.75 × 0.75 | Melt extrusion |
| 13395-20-1 | 85/15 PLGA | 5 | Span-85 | 0.75 × 0.75 | Melt extrusion |
| 13395-20-2 | 85/15 PLGA | 5 | Pluronic- | 0.75 × 0.75 | Melt extrusion |
| | | | F127 | | |
| 13395-20-3 | 85/15 PLGA | 5 | N/A | 0.75 × 0.75 | Melt extrusion |
| 13395-21-1 | D,L-PLA | 5 | mPEG | 0.75 × 0.75 | Melt extrusion |
| 13395-21-2 | 85/15 PLGA | 5 | TBO-Ac | 0.75 × 0.75 | Melt extrusion |
| 13395-24-1 | 85/15 PLGA | 5 | Span-65 | 0.75 × 0.75 | Melt extrusion |
| 13395-27-1 | 85/15 PLGA | 10 | N/A | 0.75 × 0.75 | Melt extrusion |
| 13395-27-2 | 85/15 PLGA | 15 | N/A | 0.75 × 0.75 | Melt extrusion |
| 13395-27-3 | 85/15 PLGA | 10 | Span-65 | 0.75 × 0.75 | Melt extrusion |
| 13395-27-4 | 85/15 PLGA | 10 | TBO-Ac | 0.75 × 0.75 | Melt extrusion |
| 13395-27-5 | 85/15 PLGA | 10 | Pluronic | 0.75 × 0.75 | Melt extrusion |
| | | | F127 | | |
| 13395-34-2 | D,L-PLA | 10 | N/A | 0.75 × 0.75 | Melt extrusion |
| 13395-34-3 | D,L-PLA | 10 | TBO-Ac | 0.75 × 0.75 | Melt extrusion |
| 13395-34-4 | D,L-PLA | 10 | mPEG | 0.75 × 0.75 | Melt extrusion |
| 13395-42-1 | DL-PLA/PCL | 10 | N/A | 0.75 × 0.75 | Melt extrusion |
| 13395-42-2 | DL-PLA/PCL | 15 | N/A | 0.75 × 0.75 | Melt extrusion |

TABLE 2

| | | Drug Load | | Pellet Size (L × Dia; | |
|---|---|---|---|---|---|
| Notebook ID | Polymer Type | (Wt %) | Excipient | mm) or Description | Processing |
| 13335-73-1 | POE 58 | 10 | N/A | 1.5 × 0.75 | Melt extrusion |
| 13335-73-2 | POE 58 | 20 | N/A | 0.75 × 0.75 | Melt extrusion |
| 13335-73-3 | POE 60 | 10 | N/A | 1.5 × 0.75 | Melt extrusion |
| 13335-73-4 | POE 60 | 20 | N/A | 0.75 × 0.75 | Melt extrusion |
| 13699-1-2 | POE 58 | 10 | N/A | 4 − 1.5 × 0.75 | Melt extrusion |
| 13699-1-3 | POE 58 | 20 | N/A | 1 − 0.75 × 0.75 | Melt extrusion |
| 12702-23 | tCHDM (100) | 25 | N/A | Microspheres | Double emulsion |
| 12702-26 | tCHDM/DET | 4.2 | N/A | Microspheres | Double emulsion |
| | (70/30) | | | | |
| 12702-54 | 75/25 PLGA | 20 | N/A | Microspheres | Double emulsion |
| 12702-68-9 | 75/25 PLGA | 5 | MPEG | 3 × 3 | Melt extrusion |
| 12702-68-10 | 75/25 PLGA | 5 | TBO-Ac | 3 × 3 | Melt extrusion |
| 12702-87 | 75/25 PLGA | 15 | MPEG | | Mixer-Molder |
| 12702-90 | 85/15 PLGA | 17 | N/A | | Mixer-Molder |
| 12702-78-1 | Polyketal | 7 | N/A | 2 × 3 | Melt extrusion |
| | (12833-14-1) | | | | |
| 13395-14 | 50/50 PLGA | 10 | MPEG | N/A | Melt extrusion |
| | (2A) | | | | |
| 13395-17-1 | POE (13166- | 5 | N/A | 1.5 × 1.5 | Melt extrusion |
| | 75) | | | | |
| 13395-17-2 | POE (13166- | 5 | N/A | 1.5 × 1.5 | Melt extrusion |
| | 77) | | | | |
| 13395-47-1 | DL-PCL | 10 | N/A | 1.3 × 1.3 | Melt extrusion |
| 13395-50 | DL-PCL | 10 | N/A | 1.3 × 1.3 | Melt extrusion; w/ |
| | | | | | solvent prep |
| 13395-51 | D,L-PLA | 10 | mPEG | N/A | Melt extrusion |

TABLE 3

| | | Drug Load | |
|---|---|---|---|
| Notebook ID | Polymer Type | (Wt %) | Processing |
| 00178-23 | 100 DL 5E | 8.1 | Grind drug with mortar/pestile, blend with spatula, coarsely mixed |
| 00178-15 | 100 DL 7E | 7.2 | Grind drug with mortar/pestile, blend with spatula, coarsely mixed |
| 00178-35 | 100 DL 5E | 5 | Grind drug with mortar/pestile, blend with spatula, coarsely mixed |
| 00178-16 | 100 DL 7E | 10.2 | Grind drug with mortar/pestile, blend with spatula, coarsely mixed |
| 00178-21 | 8515 DL 7E | 7.3 | Grind drug with mortar/pestile, blend with spatula, coarsely mixed |
| 00178-36 | 100 DL 7E | 5 | Grind drug with mortar/pestile, blend with spatula, coarsely mixed |
| 00178-44 | 100 DL 7E | 5.1 | Dissolved in glacial acetic acid and freeze dried |
| 00178-45 | 100 DL 7E | 4.5 | Drug and polymer blended by mortar/pestile, finely mixed, under N2 |
| 00178-63 | 100 DL 7E | 9.4 | Drug and polymer blended by mortar/pestle, finely mixed |
| 00178-08 | 100 DL 7E | 21.4 | Blend with spatula, no reduction in drug particle size |
| 00178-11 | 100 DL 7E | 7.9 | Blend with spatula, no reduction in drug particle size |
| 00178-12 | 100 DL 7E | 11.7 | Blend with spatula, no reduction in drug particle size |
| 00178-22 | 8515 DL 7E | 83.3 | Grind drug with mortar/pestile, blend with spatula, coarsely mixed |
| 00178-24 | 100 DL 5E | 10.1 | Grind drug with mortar/pestile, blend with spatula, coarsely mixed |
| tab 11 | 100 DL 5E | 5 | |
| tab 11 | 100 DL 7E | 5 | |
| tab 11 | 100 DL 5E | 5 | EtOAc coating |
| tab 11 | 100 DL 7E | 5 | EtOAc coating |
| tab 11 | 100 DL 7E | 5 | Glacial HoAc dissolution |
| tab 11 | 100 DL 7E | 5 | prepared in N2 environment |
| 00178-72 | 100 DL 7E | 4.5 | Double Extrusion (20% diluted to 5%) |
| 00178-73 | 100 DL 7E | 8.7 | Double Extrusion (20% diluted to 10%) |
| 00178-74 | 100 DLG 7E | 7.3 | API mixed with polymer using mortar/pestle |
| 00178-71 | 6535 DLG 7E | 5.3 | API mixed with polymer using mortar/pestle |
| 00178-75 | 6535 DLG 7E | 5.3 | API mixed with polymer using mortar/pestle |
| 00178-76-R1 | 100 DL 7E core with | 7.76 | coaxial extrusion, 4 different coating thicknesses |
| | 100DL coating | | |
| 00178-76-R2 | 101 DL 7E core | 6.92 | coaxial extrusion, 4 different coating thicknesses |
| | with 100DL coating | | |
| 00178-76-R3 | 102 DL 7E core | 6.76 | coaxial extrusion, 4 different coating thicknesses |
| | with 100DL coating | | |
| 00178-76-R4 | 103 DL 7E core | 8 | coaxial extrusion, 4 different coating thicknesses |
| | with 100DL coating | | |
| 00178-79-R1 | 100 DL 5E core with | 15 | coaxial extrusion, thin coat |
| | 100DL 5E coating | | |
| 00178-79-R2 | 100 DL 5E core with | 15 | coaxial extrusion, thick coat |
| | 100DL 5E coating | | |
| 00178-80-R1 | 100 DL 5E core with | 7.54 | coaxial extrusion, different coating thicknesses |
| | 100DL 5E coating | | |
| 00178-80-R2 | 100 DL 5E core with | 8.9 | coaxial extrusion, different coating thicknesses |
| | 100DL 5E coating | | |
| 00178-80-R3 | 100 DL 5E core with | 9.39 | coaxial extrusion, different coating thicknesses |
| | 100DL 5E coating | | |
| 00178-77 | 100 DL 5E | 5 | repeat of 178-35 (0.8 MM & 1.0 mm diam) |
| 00178-78 | 100 DL 5E | 5 | repeat of 178-35 (0.8 MM & 1.0 mm diam) |
| 00178-81 | 100 DL 5E | 7.2 | repeat of 178-23 |
| 00178-23B | | | EtOAc coating |
| 00178-23C | | | Polymer soln coating |

The codes within the table for the polymer are explained as follows. The first number or numbers refer to monomer mole percentage ratio of DL-lactide (e.g., polylactide) to glycolide (e.g., poly-glycolide). The letter code that follows the first number refers to the polymer(s) and is the polymer identifier. The second number, which follows the letter code for the polymer, is the target IV designator and is 10 times the midpoint of a range in dl/g. The meanings of certain IV designators are reflected in Table 4.

TABLE 4

| IV Target Designator | IV Range |
|---|---|
| 1 | 0.05-0.15 |
| 1.5 | 0.10-0.20 |
| 2 | 0.15-0.25 |
| 2.5 | 0.20-0.30 |
| 3 | 0.25-0.35 |
| 3.5 | 0.30-0.40 |
| 4 | 0.35-0.45 |
| 4.5 | 0.40-0.50 |
| 5 | 0.45-0.55 |
| 6 | 0.50-0.70 |
| 7 | 0.60-0.80 |
| 8 | 0.70-0.90 |
| 9 | 0.80-1.0 |

The final letter within the code of the polymer is the end group designator. For examples “E” refers to an ester end group, while “A” refers to an acid end group.

By way of example, 100 DL7E is a polymer that has an inherent viscosity of 0.60-0.80 dL/g. It contains 100% poly(DL-lactide) that has ester end groups. It is available from Lakeshore Biomaterials, Birmingham, Ala.

### Example 3

The efficacy of a five Month Clonidine/Polymer Drug Depot in the Rat Chronic Constriction Injury Model was evaluated. The animal model was the Bennett Model (Wistar rat). The purpose: To determine whether a five month polymer clonidine-eluting depot can improve pain associated behavioral responses in a rat model of neuropathic pain with an inflammatory component.

Experimental Design: Four loose chromic gut ligatures, 1 mm apart, were tied around the common sciatic nerve at mid-thigh. Each animal received treatment of test or control article—according to the dosing described in Table 5.

TABLE 5

| Group | | | Formulation | |
|---|---|---|---|---|
| Number | Treatment | Dose | Group | Comments |
| 1 | Clonidine | 0.02 mg/kg SC | | Clonidine control |
| 2 | 100 DL 7E | 0% | | 4 pellets (3 mm × 0.7 mm) |
| 3 | 100 DL 7E | 5% | | Clonidine HCl; 4 pellets |
| | | | | (3 mm × 0.7 mm) |
| 4 | 100 DL 5E | 5% | | 3 pellets (3 mm × 0.7 mm) |
| 5 | 100 DL 5E | 7% | | 3 pellets (3 mm × 0.7 mm) |
| 6 | 100 DL 7E | 7% | | 3 pellets (3 mm × 0.7 mm) |
| 7 | POE | 0% | | 5 pellets (1.5 mm × 0.7 mm) |
| 8 | POE | 10 and 20% | | clonidine-base; 5 pellets |
| | | | | (1 20% @ 0.7 mm^{2;} |
| | | | | 4 10% @ 1.5 mm × 0.7 mm) |

The present study was conducted for a period of 64 days and employed the following two tests: (1) the Hargreaves test; and (2) the von Frey test. The Hargreaves Tests of Thermal Hyperalgesia were conducted on days 7, 14, 21, 28, 35, 42, 49, 56 and 63. The von Frey monofilament test of mechanical allodynia (performed the day following Thermal testing) were conducted on days-8, 15, 22, 29, 36, 43, 50, 57 and 64. The results of these tests show the efficacy of clonidine of the recited time periods.

The pain behavioral response (measured as a percentage of baseline) for thermal hyperalgesia indicates that clonidine delivered subcutaneously at 0.02 mg/kg/day consistently reduced the behavioral response when compared to either unloaded polymer depots (100 DL 7W Control or POE Control) (58% vs. 45%). All five clonidine-loaded polymer depots were able to reduce pain behavioral responses when compared to unloaded depot; although, each formulation experienced a drop in efficacy at some point after the initial burst of drug at implantation. The pain behavioral response (measured as a percentage of baseline) for mechanical allodynia indicates that clonidine delivered subcutaneously at 0.02 mg/kg/day reduced the behavioral response when compared to either unloaded polymer depots (100 DL 7W Control or POE Control). The pain results indicate that the clonidine may be useful in treating periodontal disease. The clonidine depot would be expected to reduce inflammation, reduce bone loss and reduce pain due to periodontal disease.

### Example 4

### Exemplary Sulindac formulations

A number of formulations were created and are summarized in Table 6, Table 7 and Table 8 below.

TABLE 6
First set of exemplary sulindac formulations

| | Drug Load |
|---|---|
| Polymer | (wt %) |
| 100 DL 7E | 14.2 |
| 100 DL 7E | 20.4 |
| 8515 DLG 7E | 14.4 |
| 8515 DLG 7E | 19.2 |
| 100 DL 5E | 14.8 |
| 100 DL 5E | 19 |
| 100 DL 7E | 30.7 |
| 100 DL 7E | 40.7 |
| 100 DL 7E | 19.2 |
| 100 DL 7E | 27.1 |
| 8515 DLG 7E | 28.8 |

The codes within table 6 for the polymer are explained as follows. The first number or numbers refers to monomer mole percentage ratio of DL-lactide (e.g., polylactide) to glycolide (e.g., poly-glycolide). The letter code that follows the first number refers to the polymer(s) and is the polymer identifier. The second number, which follow the letter code for the polymer, is the target IV designator and is 10 times the midpoint of a range in dl/g. The meanings of certain IV designators are reflected in Table 7.

TABLE 7

| IV Target Designator | IV Range |
|---|---|
| 1 | 0.05-0.15 |
| 1.5 | 0.10-0.20 |
| 2 | 0.15-0.25 |
| 2.5 | 0.20-0.30 |
| 3 | 0.25-0.35 |
| 3.5 | 0.30-0.40 |
| 4 | 0.35-0.45 |
| 4.5 | 0.40-0.50 |
| 5 | 0.45-0.55 |
| 6 | 0.50-0.70 |
| 7 | 0.60-0.80 |
| 8 | 0.70-0.90 |
| 9 | 0.80-1.0 |

The final letter within the code of the polymer is the end group designator. For examples “E” refers to an ester end group, while “A” refers to an acid end group.

By way of example, 100 DL 7E is a polymer that has an inherent viscosity of 0.60-0.80 dL/g. It contains 100% poly(DL-lactide) that has ester end groups. It is available from Lakeshore Biomaterials, Birmingham, Ala.

Table 8 shows a set of exemplary sulindac formulations.

TABLE 8

| Notebook | | Drug Load | | Pellet Size (L × Dia; mm) | |
|---|---|---|---|---|---|
| ID | Polymer Type | (WT %) | Excipient | or Description | Processing |
| 12702-28-1 | 85/15 PLGA & Sul particles | 30 | N/A | Drug loaded particles in gel | Hand-Mix |
| | Suspended in PDE gel (13027- | 3 | | | |
| | 34) | | | | |
| 12702-28-3 | POE gel (13027-34) | 3 | N/A | Free drug suspended in gel | Hand-Mix |
| 12702-28-4 | POE gel (13027-34) | 6 | N/A | Free drug suspended in gel | Hand-Mix |
| 12702-30-1 | POE (12811-01) | 19 | N/A | Drug loaded MS suspended in gel | Hand-Mix |
| | Microparticles | | | | |
| | POE gel (13027-34) | | | | |
| 12702-31-1 | 85/15 PLGA | 1.5 | N/A | Drug loaded into polymer/solvent | Hand-Mix |
| 12702-31-2 | 85/15 PLGA | 3 | N/A | Drug loaded into polymer/solvent | Hand-Mix |
| 12702-32 | POE (12811-01) Microparticles | 0.4 | N/A | Drug loaded MS suspended in gel | Hand-Mix |
| | POE gel (13027-34) | | | | |
| 12702-36-1 | POE 12811-01 & 13027-34 | 10 | MgCO3 | Drug loaded particles suspended in gel | Hand-Mix |
| 12702-36-2 | POE 12811-01 & 13027-34 | 15 | MgCO3 | Drug loaded particles suspended in gel | Hand-Mix |
| 12702-36-3 | POE 12811-01 & 13027-34 | 20 | MgCO3 | Drug loaded particles suspended in gel | Hand-Mix |
| 12702-38 | POE (12811-01) Microparticles | 20 | MgCO3 | Drug loaded particles suspended in gel | Hand-Mix |
| | POE gel 13027-40 | | | | |
| 12702-39 | POE 13027-34 | 0.6 | MgCO3 | Free drug suspended in gel | Hand-Mix |
| 12702-40 | POE (12811-01) Micropartices | 0.4 | MgCO3 | Drug loaded MS suspended in gel | Hand-Mix |
| | POE 13027-32 | | | | |
| 12702-41 | POE (12811-01) Micropartices | 0.4 | MgCO3 | Drug loaded MS suspended in gel | Hand-Mix |
| | POE 13027-32 | | | | |
| 12702-42-1 | 75/25 PLGA | 15 | mPEG | 10 × 1.5 | Solvent |
| | | | | | prep |
| | | | | | followed by |
| | | | | | extrusion |
| 12702-49 | 50/50 PLGA | 15 | PEG | 10 × 1.5 | Solvent-less |
| | | | | | prep |
| | | | | | followed by |
| | | | | | extrusion |
| 12702-57-1 | 75/25 PLGA | 11 | Parafin oil | N/A; no product | Polymer |
| | | | | | dissolved in |
| | | | | | NMP |
| 12702-57-2 | 75/25 PLGA | 10 | Glycerol Mono | | Polymer |
| | | | | | dissolved in |
| | | | | | NMP |
| 12702-57-3 | 75/25 PLGA | 11 | PEG bis(ethyl | | Polymer |
| | | | hex) | | dissolved in |
| | | | | | NMP |
| 12702-57-4 | 75/25 PLGA | 10 | | | Polymer |
| | | | | | dissolved in |
| | | | | | NMP |
| 12702-60-1 | 75/25 PLGA | 15 | N/A | Ground Particles | Solvent prep |
| | | | | | followed by |
| | | | | | grinding |
| 12702-66-1 | 75/25 PLGA-PEG-2000 | 5 | mPEG | 10 × 2 | Solvent prep |
| | | | | | followed by |
| | | | | | extrusion |
| 12702-66-2 | 75/25 PLGA-PEG-2000 | 5 | TBO-Ac | 10 × 2 | Solvent prep |
| | | | | | followed by |
| | | | | | extrusion |
| 12702-66-3 | 75/25 PLGA-PEG-6000 | 5 | mPEG | 10 × 2 | Solvent prep |
| | | | | | followed by |
| | | | | | extrusion |
| 12702-66-4 | 75/25 PLGA-PEG-6000 | 5 | TBO-Ac | 10 × 2 | Solvent prep |
| | | | | | followed by |
| | | | | | extrusion |
| 12702-66-5 | 75/25 PLGA | 5 | mPEG | 10 × 1.5 | Solvent prep |
| | | | | | followed by |
| | | | | | extrusion |
| 12702-66-6 | 75/25 PLGA | 5 | TBO-Ac | 10 × 2 | Solvent prep |
| | | | | | followed by |
| | | | | | extrusion |
| 12702-66-7 | 75/25 PLGA (HMW) | 5 | mPEG | 10 × 2 | Solvent prep |
| | | | | | followed by |
| | | | | | extrusion |
| 12702-66-8 | 75/25 PLGA (HMW) | 5 | TBO-Ac | 10 × 1.5 | Solvent prep |
| | | | | | followed by |
| | | | | | extrusion |
| 12702-66-14 | 75/25 PLGA | 5 | mPEG | 10 × 2 | Solvent prep |
| | | | | | followed by |
| | | | | | extrusion |
| 12702-72-4 | 85/15 PLGA | 5 | mPEG | 10 × 1.5 | Solvent prep |
| | | | | | followed by |
| | | | | | extrusion |
| 12702-75-1 | 85/15 PLGA | 5 | N/A | 10 × 1 | Solvent-less |
| | | | | | prep |
| | | | | | followed by |
| | | | | | extrusion |
| 12702-75.2 | 85/15 PLGA | 10 | N/A | 10 × 1 | Solvent-less |
| | | | | | prep |
| | | | | | followed by |
| | | | | | extrusion |
| 12702-75-3 | 85/15 PLGA | 5 | mPEG | 10 × 1 | Solvent-less |
| | | | | | prep |
| | | | | | followed by |
| | | | | | extrusion |
| 12702-75-4 | 85/15 PLGA | 10 | mPEG | 10 × 1 | Solvent-less |
| | | | | | prep |
| | | | | | followed by |
| | | | | | extrusion |
| 12702-79-1 | 85/15 PLGA | 5 | N/A | 0.8 × 0.8 | Solvent-less |
| | | | | | prep |
| | | | | | followed by |
| | | | | | extrusion |
| 12702-79.2 | 85/15 PLGA | 5 | N/A | 0.8 × 0.8 | Solvent-less |
| | | | | | prep |
| | | | | | followed by |
| | | | | | extrusion |
| 12702-79-3 | 85/15 PLGA | 5 | mPEG | 0.8 × 0.8 | Solvent-less |
| | | | | | prep |
| | | | | | followed by |
| | | | | | extrusion |
| 12702-79-4 | 85/15 PLGA | 5 | mPEG | 0.8 × 0.8 | Solvent-less |
| | | | | | prep |
| | | | | | followed by |
| | | | | | extrusion |

Sulindac drug loads ranged from 0.4 wt % to 30 wt. %. The polymers included PLGA 85/15, 75/25, or 50/50, DL, POE, or DL-PLA. The excipients include mPEG, PEG, barium sulfate with mPEG, magnesium carbonate, paraffin oil, PEG, glycerol monooleate, TBO-AC, or no excipients. Excipients may be added in weight percentages from 0.001 wt % or 0.01 wt. % to 50 wt. %, 5 to 25 wt %, or 5 to 12 wt %.

In vitro elution profiles were obtained from about 3 to 150 days, which may be useful for treating periodontal disease are shown in Table 9.

TABLE 9

| | | Drug Load |
|---|---|---|
| Batch Number | Polymer | (wt %) |
| 00178-13 | 100 DL 7E | 14.2 |
| 00178-28 | 100 DL 7E | 20.4 |
| 00178-17 | 8515 DLG 7E | 14.4 |
| 00178-18 | 8515 DLG 7E | 19.2 |
| 00178-19 | 100 DL 5E | 14.8 |
| 00178-20 | 100 DL 5E | 19 |
| 00178-46 | 100 DL 5E | 30.7 |
| 00178-47 | 100 DL 7E | 40.7 |
| 00178-48 | 100 DL 7E | 19.2 |
| 00178-49 | 100 DL 7E | 27.1 |
| 00178-50 | 8515 DLG 7E | 28.8 |

A separate batch of sulindac formulations (shown in table 9) and their respective In Vitro elution profiles are shown ranging from about 40 to 90 days, which may be useful for treating periodontal disease.

### Example 5

### Testing in Rats

The efficacy of a 5 Month Sulindac/Polymer Drug Depot in the Rat Chronic Constriction Injury Model was evaluated. The animal model was the Bennett Model (Wistar rat). The purpose: To determine whether a 5 month polymer sulindac-eluting depot can improve pain associated behavioral responses in a rat model of neuropathic pain.

Experimental Design: Four loose chromic gut ligatures, 1 mm apart, were tied around the common sciatic nerve at mid-thigh. Each animal received treatment of test or control article—according to the dosing described in Table 10.

TABLE 10

| Group Number | Treatment |
|---|---|
| 1 | Sulindac 0.4 mg/kg |
| 2 | 85/15 PLGA Control |
| 3 | 85/15 PLGA-20% |
| 4 | 85/15 PLGA-27% |
| 5 | DL-PLA-25% |
| 6 | 100-DL-7E-30% |
| 7 | 100-DL-7E-20% |
| 8 | 85/15 PLGA-30% |

To date, the present study was conducted for a period of 57 days and employed the following two tests: (1) the Hargreaves test on days 7, 14, 21, 28, 35, 42, 49, and 56; and (2) the von Frey test on days 8, 15, 22, 29, 36, 43, 50, and 57. The results of these tests show the efficacy of sulindac of the recited time periods.

The pain behavioral response (measured as a percentage of baseline) for thermal hyperalgesia indicates that sulindac delivered intraperitoneally at 0.4 mg/kg/day consistently reduced the behavioral response when compared to unloaded polymer depot (85/15 PLGA Control). All six sulindac-loaded polymer depots were able to reduce pain behavioral responses, at least for one time point, when compared to unloaded depot. Most of the formulation experienced a drop in efficacy at some point after the initial burst of drug at implantation; however, the 27% 85/15 PLGA and 25% DL-PLA formulations took a few weeks to reduce pain at all. The pain induced by periodontal disease is of an inflammatory nature.

The pain behavioral response (measured as a percentage of baseline) for mechanical allodynia indicates that sulindac delivered intraperitoneally at 0.4 mg/kg/day reduced the behavioral response when compared to unloaded polymer depots (85/15 PLGA Control). All six sulindac-loaded polymer depots were able to reduce pain behavioral responses when compared to unloaded depot; although, each formulation took a few weeks to demonstrate this effect. These results show that sulindac at the different drug loads can reduce, prevent, or treat pain for time periods beyond 57 days and may be useful for treating periodontal disease.

Certain abbreviations are used in the some of the tables and figures. The abbreviation. The abbreviations “DL” or “DL-PLA” refer to poly(DL-lactide). The abbreviation “PLGA” refers to poly(lactide-co-glycolide).

Often times when the polymer is a heteropolymer or copolymer, there is a mixture of monomer species in the polymer. The mole ratio may be indicated and varied from 0:100 to 100:0 and ranges in between these mole ratios. For example, 85:15 PLGA, the 85 refers to the monomer mole % 85 of DL (poly DL-lactide) in the polymer, while the 15 refers to the mole percent of the PGA (polyglycolide) in the polymer.

The codes within the table for the polymer are explained as follows. The first number or numbers refers to monomer mole percentage ratio of DL-lactide (e.g., polylactide) to glycolide (e.g., poly-glycolide). The letter code that follows the first number refers to the polymer(s) and is the polymer identifier. The second number, which follows the letter code for the polymer, is the target IV designator and is 10 times the midpoint of a range in dl/g. The meanings of certain IV designators is reflected in Table A below.

TABLE A

| IV Target Designator | IV Range |
|---|---|
| 1 | 0.05-0.15 |
| 1.5 | 0.10-0.20 |
| 2 | 0.15-0.25 |
| 2.5 | 0.20-0.30 |
| 3 | 0.25-0.35 |
| 3.5 | 0.30-0.40 |
| 4 | 0.35-0.45 |
| 4.5 | 0.40-0.50 |
| 5 | 0.45-0.55 |
| 6 | 0.50-0.70 |
| 7 | 0.60-0.80 |
| 8 | 0.70-0.90 |
| 9 | 0.80-1.0 |

The final letter within the code of the polymer is the end group designator. For examples “E” refers to an ester end group, while “A” refers to an acid end group.

The polymers may have different end groups such as acid and ester end groups. Implantable elastomeric depot compositions having a blend of polymers with different end groups are used the resulting formulation will have a lower burst index and a regulated duration of delivery. For example, one may use polymers with acid (e.g., carboxylic acid) and ester end groups (e.g., lauryl, methyl, and/or ethyl ester end groups).

By way of example, 100 DL 7E is a polymer that has an inherent viscosity of 0.60-0.80 dL/g. It contains 100% poly(DL-lactide) that has ester end groups. It is available from Lakeshore Biomaterials, Birmingham, Ala.

### Fluocinolone

### Example 6

Table 11 provides a table fourteen formulations that contain fluocinolone and excipients, including one formulation that contains no excipients.

TABLE 11

| | | | Drug Load |
|---|---|---|---|
| Batch Number | Polymer | Excipient | (wt %) |
| 00178-27 | 100 DL 7E | 5% PEG 1500 | 1.71 |
| 00178-28 | 100 DL 7E | 5% Pluronic F-68 | 1.79 |
| 00178-29 | 100 DL 7E | 7% 5050 DL 7A | 1.98 |
| 00178-30 | 100 DL 5E | 5% PEG 1500 | 1.97 |
| 00178-31 | 100 DL 5E | 5% Pluronic F-68 | 1.82 |
| 00178-32 | 100 DL 5E | 7% 5050 DL 7A | 1.94 |
| 00178-33 | 100 DL 5E | 7% 5050 DL 7A | 1.98 |
| 00178-34 | 100 DL 7E | | 1.92 |
| 00178-38 | 100 DL 5E | 10% PEG 1500 | 0.96 |
| 00178-39 | 100 DL 5E | 7% 5050 7A | 0.89 |
| | | 5% PEG 1500 | |
| 00178-40 | 100 DL 5E | 10% 5050 7a | 0.98 |
| 00178-41 | 100 DL 7E | 7% 5050 7A | 0.81 |
| 00178-42 | 100 DL 7E | 10% PEG 1500 | 0.85 |
| 00178-54 | 100 DL 5E | 10% PEG 1500 | 0.88 |

The cumulative API released was 0.01 to 0.08 micrograms daily for at least 2 weeks to as long as 70 days. The % drug load released during the period was up to 50% to 65% percent of drug released from the depot.

The In-Vitro Elution Studies were carried out at 37° C. in phosphate-buffered saline with 0.5% SDS (pH 7.4). Briefly, the rods (n=3) were weighed prior to immersion in 10 mL of PBS. At regular time intervals, the PBS was removed for analysis and replaced with 10 mL of fresh PBS. The PBS-elution buffer was analyzed for clonidine content using UV-Vis spectrometry.

### Example 7

A 2-month chronic constriction injury (CCI) model of neuropathic pain was used to evaluate different formulations of a corticosteroid, fluocinolone, encapsulated in bioerodable polymers compared to fluocinolone given subcutaneously (SC). Different formulations as provided in Table B below were evaluated for reducing pain-associated behaviors: Thermal paw withdrawal latency was evaluated at baseline 7, 14, 21, 28, 35, 42, 49, and 56 days post-operatively, while mechanical threshold was evaluated at 8, 15, 22, 29, 36, 43, 50, and 57 days post-operatively. Fluocinolone reduced pain threshold, which may be useful in periodontal disease. The pain of periodontal disease is due to the inflammation. Fluocinolone is useful both in reducing pain and reducing the inflammation that causes pain.

There were seven groups of animals tested. Each animal received treatment of test or control article according to the dosing groups (n=8) described in the Table B. Group 1 received daily drug injections. Groups 2-7 received solid, polymer implants that were implanted caudal to the CCI in a manner that totally surrounds the nerve.

TABLE B

| Group | | | |
|---|---|---|---|
| Number | Treatment | Dose | Comments |
| 1 | Fluocinolone | 0.5 ug/kg | Daily SC |
| | | | administration |
| 2 | 85/15 PLGA | 0% | Control |
| 3 | 100 DL 5E + Glacial acetic | 0% | Control |
| | acid/7% 5050 7A | | |
| 4 | 85/15 PLGA + 10% mPEG | 20% | 1 pellet |
| 5 | 100 DL 5E + 10% PEG1500 | 1% | 1 pellet |
| 6 | 100 DL 7E + 5% PEG/7% | 1% | 1 pellet |
| | 5050 7A | | |
| 7 | 100 DL 7E + 10% PEG1500 | 1% | 1 pellet |

Mechanical allodynia was measured using von Frey monofilaments (Stoelting, Wood Dale, Ill.) with varying stiffness (2.0-15.0 g) on Days 1, 8, 15, 22, 29, 36, 43, 50 and 57 as described previously. Animals were placed on a perforated metallic platform and allowed to habituate to their surroundings for a minimum of 15 minutes before testing. The 50% paw withdrawal threshold response was determined by a sequential increasing and/or decreasing of the stimulus strength (the “up-down method” of Dixon). Each filament was applied with enough pressure to cause a buckling effect. Absence of a paw lifting/withdrawal response after 5 seconds prompted the use of the filament of next higher weight. Paw withdrawal indicating a positive response prompted the use of a weaker filament. After the initial response (i.e., paw withdrawal), the testing continued for four additional measurements and was used to calculate the response threshold. Four consecutive positive responses received a score of 0.25 g, and five consecutive negative responses (i.e., no paw withdrawal) received a score of 15 g. The 50% paw withdrawal threshold was calculated using the formula: 10 (Xf+kd)/10,000, where Xf is the final von Frey filament used (log units), k is a value that analyzes the response pattern (taken from the table published by Chaplan et al.), and d is the mean difference between stimuli (log units). The mean and standard error of the mean (SEM) were determined for each treatment group.

The results showed that fluocinolone drug depots were effective at reducing pain and/or inflammation when compared to the control (unloaded polymer depots) for at least 57 days. Thus, fluocinolone may be useful in treating periodontal disease.

### Example 8

### Fluocinolone Formulations and Release Profiles

Fluocinolone is a potent steroid with glucocorticoid activity. To get consistent release additional excipients were added to the polymer formulation. For example with drug loads of 1% to 20% fluocinolone, 85/15 PLGA or DL-PLA or DL-PLA and 50/50 PLGA mixture can be added in an amount of from about 10% to 98%. The depot can be extruded and made into different sizes (e.g., 0.75 (length)×0.75 mm (diameter), 0.8×0.8 mm, 1×1 mm pellet sizes).

Table C shows the various fluocinolone formulations made.

TABLE C

| | | Drug | | Pellet Size (L × Dia; | |
|---|---|---|---|---|---|
| Notebook | Polymer | Load | | mm) | |
| ID | Type | (WT %) | Excipient | or Description | Processing (elution = x) |
| 13395-3-4 | 85/15 | 5 | mPEG | N/A | Melt processed |
| | PLGA | | | | |
| 13395-4-1 | 85/15 | 10 | mPEG | 0.75 × 0.75 | Melt processed (x) |
| | PLGA | | | | |
| 13395-4-2 | 85/15 | 15 | mPEG | 0.75 × 0.75 | Melt processed (x) |
| | PLGA | | | | |
| 13395-4-3 | 85/15 | 20 | mPEG | 0.75 × 0.75 | Melt processed (x) |
| | PLGA | | | | |
| 13395-35-5 | DL-PLA: | 10 | mPEG | 0.8 × 0.8 | Melt processed (x) |
| | 50/50 | | | | |
| | PLGA | | | | |
| 13395-35-6 | 85/15 | 10 | mPEG | 0.8 × 0.8 | Melt processed |
| | PLGA | | | | |
| | 50/50 | | | | |
| | PLGA | | | | |
| 13395-39-3 | 85/15 | 10 | mPEG | 0.8 × 0.8 | Melt processed |
| | PLGA | | | | |
| 13395-42-3 | 85/15 | 5 | D-sorbitol | 0.75 × 0.75 | Melt processed (x) |
| | PLGA | | | | |
| 13395-42-4 | 85/15 | 5 | D-sorbitol; | 0.75 × 0.75 | Melt processed |
| | PLGA | | mPEG | | |
| 13395-42-5 | 85/15 | 5 | mPEG | 0.75 × 0.75 | Melt processed |
| | PLGA | | | | |
| 13395-42-6 | 85/15 | 5 | D-sorbitol | | |
| | PLGA | | | | |
| 13395-48-1 | 85/15 | 5 | Maltodextrin; | 0.75 × 0.75 | Melt processed (x) |
| | PLGA | | mPEG | | |
| 13395-53-1 | DL-PLA | 5 | mPEG | 1 × 1 | Melt processed |
| 13395-53-2 | DL-PLA | 5 | D-sorbitol; | 1 × 1 | Melt processed |
| | | | mPEG | | |
| 13395-53-3 | DL-PLA | 5 | N/A | 1 × 1 | Melt processed |
| 13395-53-4 | DL-PLA | 5 | D-sorbitol | 1 × 1 | Melt processed |
| 13395-54-1 | DL-PLA | 5 | Maltodextrin; | 1 × 1 | Melt processed |
| | | | mPEG | | |
| 13395-54-3 | DL-PLA | 5 | 10% | 1 × 1 | Melt processed |
| | | | Maltodextrin; | | |
| | | | mPEG | | |
| 13395-54-6 | 85/15 | 20 | mPEG | 0.75 × 0.75 | Melt processed (x) |
| | PLGA | | | | |
| 13395-59-1 | DL-PLA | 10 | Maltodextrin | N/A | Melt processed |
| 13395-63-6 | 85/15 | 20 | mPEG | 0.75 × 0.75 | Melt processed (x) |
| | PLGA | | | | |
| 13395-66-1 | DL-PA | 3 | 40% PEG | N/A | Melt processed |
| | | | (3350 MW) | | |
| 13395-72 | 85/15 | 2 | B- | 0.75 × 0.75 | Melt processed (x) |
| | PLGA | | cyclodextrin | | |
| | | | & mPEG | | |
| 13395-72 | 85/15 | 2 | B- | 0.75 × 0.75 | Melt processed |
| | PLGA | | cyclodextrin | | |
| | | | & mPEG | | |

Some of the formulation's elution profiles were tested and ranged from 38 days to 100 days with fluocinolone release rates of 1% to 60% cumulative release over 20, 40, 50, 80, and 100 days. These types of release profiles would be beneficial in reducing, treating or preventing periodontal disease.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. An implantable drug depot for reducing, preventing or treating periodontal disease in a patient in need of such treatment, the implantable drug depot comprising clonidine in an amount from about 1 wt. % to about 20 wt. % of the drug depot, fluocinolone in an amount from about 0.05 wt. % to about 25 wt. % of the drug depot, and/or sulindac in an amount from about 5 wt. % to about 40 wt. % of the drug depot, and at least one biodegradable polymer, wherein the drug depot is capable of releasing clonidine, fluocinolone and/or sulindac over a period of at least two weeks.

2. An implantable drug depot according to claim 1, wherein the clonidine comprises from about 5 wt. % to about 15 wt. % of the drug depot, the sulindac comprises from about 5 wt. % to about 15 wt. % of the drug depot; and/or the fluocinolone comprises from about 1 wt. % to about 15 wt. % of the drug depot.

3. An implantable drug depot according to claim 1, wherein said at least one biodegradable polymer comprises at least 80 wt. % to 90 wt. % of the drug depot.

4. An implantable drug depot according to claim 1, wherein the at least one biodegradable polymer comprises one or more of poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone or a combination thereof.

5. An implantable drug depot according to claim 4, wherein the at least one biodegradable polymer comprises poly(lactic-co-glycolide) and said poly(lactic-co-glycolide) comprises a mixture of polyglycolide and polylactide.

6. An implantable drug depot according to claim 5, wherein said mixture comprises more polylactide than polyglycolide.

7. An implantable drug depot according to claim 1, wherein the clonidine comprises clonidine hydrochloride, the sulindac comprises sulindac sodium, and/or the fluocinolone comprises fluocinolone acetonide.

8. An implantable drug depot according to claim 1, wherein the drug depot releases: (i) a bolus dose of the clonidine, sulindac, and/or fluocinolone at a site in the oral cavity and/or (ii) an effective amount of the clonidine, sulindac, and/or fluocinolone over a period of at least two weeks to two months.

9. An implantable drug depot according to claim 1, wherein the at least one biodegradable polymer comprises poly(lactic-co-glycolide) or poly(orthoester) or a combination thereof, and said at least one biodegradable polymer comprises at least 80 wt. % of said drug depot.

10. An implantable drug depot for reducing, preventing or treating periodontal disease in a patient in need of such treatment, the implantable drug depot comprising clonidine hydrochloride in an amount of from about 1 wt. % to about 20 wt. % of the drug depot, fluocinolone acetonide in an amount from about 0.05 wt. % to about 25 wt. % of the drug depot, and/or sulindac sodium in an amount from about 20 wt. % to about 40 wt. % of the drug depot, and at least one polymer, wherein the at least one polymer comprises one or more of poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone or a combination thereof.

11. An implantable drug depot according to claim 10, wherein said at least one biodegradable polymer comprises at least 80 wt. % to 90 wt. % of the drug depot.

12. An implantable drug depot according to claim 10, wherein the at least one biodegradable polymer comprises and said poly(lactic-co-glycolide) comprises a mixture of polyglycolide and polylactide.

13. An implantable drug depot according to claim 12, wherein said mixture comprises more polylactide than polyglycolide.

14. A method for treating periodontal disease in a patient in need of such treatment, the method comprises implanting into an oral cavity of the patient a drug depot comprising clonidine in an amount of from about 1 wt. % to about 20 wt. % of the drug depot, fluocinolone in an amount from about 0.05 wt. % to about 25 wt. % of the drug depot, and/or sulindac in an amount from about 5 wt. % to about 40 wt. % of the drug depot, and at least one biodegradable polymer, wherein the drug depot is capable of releasing clonidine, fluocinolone and/or sulindac over a period of at least two weeks.

15. A method according to claim 14, wherein said clonidine comprises from about 5 wt. % to about 15 wt. % of the drug depot, the fluocinolone comprises from about 1 wt. % to about 15 wt. % of the drug depot, and/or the sulindac comprises from about 5 wt. % to about 15 wt. % of the drug depot.

16. A method according to claim 14, wherein the clonidine comprises clonidine hydrochloride, the sulindac comprises sulindac sodium, and/or the fluocinolone comprises fluocinolone acetonide.

17. A method according to claim 14, wherein said biodegradable polymer comprises at least 80 wt. % to 90 wt. % of the drug depot.

18. A method according to claim 14, wherein the at least one biodegradable polymer comprises one or more of poly(lactide-co-glycolide) (PLGA), polylactide (PLA), polyglycolide (PGA), D-lactide, polyorthoester (POE), D,L-lactide, L-lactide, D,L-lactide-caprolactone, D,L-lactide-glycolide-caprolactone or a combination thereof.

19. A method according to claim 18, wherein the at least one biodegradable polymer comprises poly(lactic-co-glycolide) and said poly(lactic-co-glycolide) comprises a mixture of polyglycolide and polylactide.

20. A method according to claim 14, wherein the implanting comprises administering one or more drug depots at a plurality of sites that triangulate a periodontal diseased tissue in of the oral cavity.
